(19) United States
(12) Patent Application Publication
(10) Pub. No.: US 2023/0152226 A1
(43) Pub. Date: May 18, 2023

(54) OPTICAL SENSORS

(71) Applicant: LumiraDx UK Ltd., London (GB)

(72) Inventors: Stephen V. Fiacco, South Pasadena, CA (US); Amanda N. Bruin, Solana Beach, CA (US); Matthew J. Willmore, Solana Beach, CA (US); Tristin E. Rose, Solana Beach, CA (US); Guillermo Martinez-Ariza, Solana Beach, CA (US); Keerthi Boddupally, San Diego, CA (US)

(21) Appl. No.: 17/949,073

(22) Filed: Sep. 20, 2022

Related U.S. Application Data
(63) Continuation-in-part of application No. PCT/US2021/023530, filed on Mar. 22, 2021.
(60) Provisional application No. 62/992,511, filed on Mar. 20, 2020.

(51) Int. Cl. G01N 21/64; B01L 3/00

(52) U.S. Cl. CPC G01N 21/6428; B01L 3/502761; G01N 2021/6439; B01L 2300/0654; B01L 2200/16; B01L 2200/0652

(57) ABSTRACT

An optical sensor is used to detect a target in a sample liquid. The optical sensor has an optical sensing layer including, in the same layer, a fluorescent reporter, a fluorescent reference, and an optical isolating reagent. The optical sensing layer has an outer surface that contacts the sample liquid and an opposing surface through which a light source irradiates the optical sensing layer with excitation light, and a detector detects fluorescence emitted from within the optical sensing layer. The optical isolating reagent reduces the amount of (i) excitation light that passes through the optical sensing layer and reaches the sample liquid and (ii) background fluorescence that is emitted within the sample liquid and passes back through the optical sensing layer to the detector. Accordingly, the optical reporter and optical reference fluorescence can be detected with higher signal to noise ratios than in the absence of the optical isolating reagent.

### RELATED APPLICATIONS

The present application is a continuation-in-part of International (PCT) Patent Application No. PCT/US2021/023530, filed Mar. 22, 2020, which claims the benefit of and priority to U.S. Provisional Patent Application No. 62/992,511 filed Mar. 20, 2020. Each of the foregoing applications is incorporated by reference herein in its entirety.

### FIELD OF THE INVENTION

The present invention relates to optical sensors.

### BACKGROUND

Optical sensors may be used to determine the presence and/or amount of a target present in a sample liquid. Exemplary targets include biological molecules such as proteins or small molecules, and inorganic targets such as physiologically important ions. A sensor may include an optical reporter that provides an optical reporter signal, e.g., fluorescence, indicative of the presence and/or amount of the target. For example, the amount of fluorescence emitted by an optical reporter may change and/or shift in wavelength in the presence of the target. Alternatively, or in addition, the optical reporter signal may be indicative of the presence and/or amount of an indicator species that itself is indicative of the presence and/or amount of target. For example, the target may be a substrate of an enzyme and the indicator species may be an enzymatic reaction product produced by the enzyme in the presence of the optical reporter. An optical sensor may also include an optical reference that provides an optical reference signal, e.g., fluorescence, generally independent of the presence and/or amount of target or indicator species. The optical reference signal may be used to adjust or correct the optical reporter signal for, e.g., variations in the amounts of reagents or sensor response.

### SUMMARY OF THE INVENTION

In embodiments, an optical reporter providing an optical reporter signal, e.g., fluorescence, indicative of the presence and/or amount of sodium ion (Na⁺) includes a compound having the general formula:

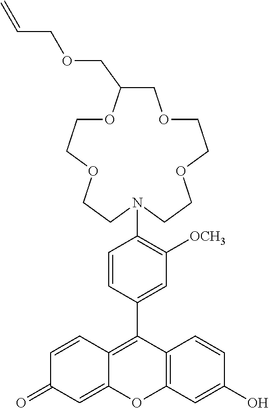

In embodiments, an optical reporter providing an optical reporter signal, e.g., fluorescence, indicative of the presence and/or amount of potassium ion (K⁺) includes a compound having the general formula:

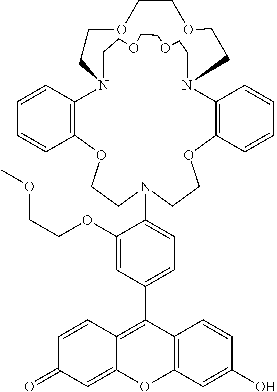

In embodiments, an optical reporter providing an optical reporter signal, e.g., fluorescence, indicative of the presence and/or amount of potassium ion (K⁺) includes a compound having the general formula:

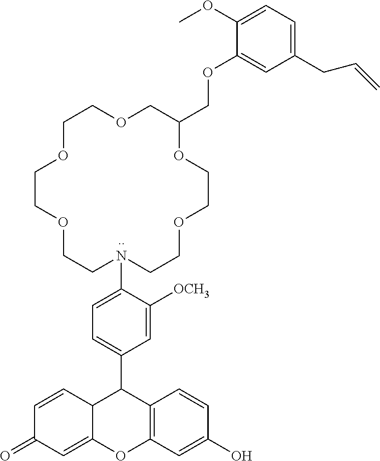

In embodiments, an optical reporter providing an optical reporter signal, e.g., fluorescence, indicative of the presence and/or amount of calcium ion (Ca⁺⁺) includes a compound having the general formula:

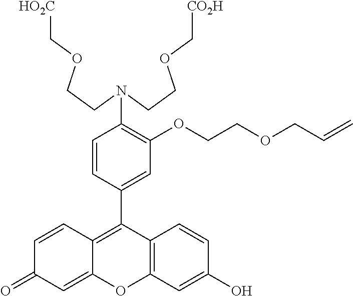

In embodiments, an optical reporter providing an optical reporter signal, e.g., fluorescence, indicative of the presence and/or amount of hydrogen ion (H⁺), e.g., a pH sensor, includes a compound having the general formula:

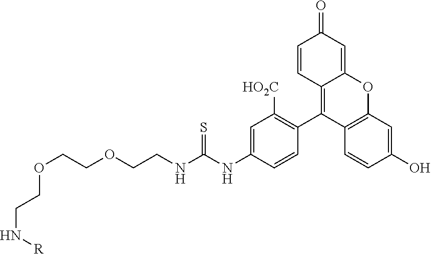

In embodiments, including the embodiments illustrated above, an optical reporter includes a first portion (moiety) configured to interact with (e.g., chelate or bind) a target, and a second portion (moiety) that emits fluorescence, wherein the intensity of the fluorescence changes upon intersection with target. In embodiments, the first portion (moiety) is a ring structure configured to chelate a target. In embodiments, detection of the change in intensity of the fluorescence indicates the presence of the target, e.g., in a sample.

In embodiments, an optical reporter includes a derivative of any of the forgoing optical reporters. In some embodiments, the derivative includes one of the forgoing optical reporters with one or more hydrogen atoms substituted with a group selected from an aliphatic group, a lipophilic group, a hydrophilic group, an electron withdrawing group, an electron donating group, or a combination thereof. For example, a derivative of any of the forgoing optical reporters may include an optical reporter substituted with such a group or groups, e.g., a halogen, at the 2 and/or 7 position of the xanthene group of the optical reporter. An example of such a derivative is an optical reporter providing an optical reporter signal, e.g., fluorescence, indicative of the presence and/or amount of calcium ion (Ca⁺⁺) includes a compound having the general formula and a fluorine substituted for hydrogen at the 2 and/or 7 position of the xanthene group of the optical reporter:

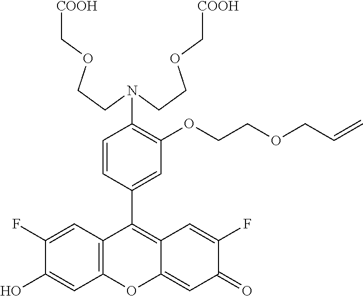

In some embodiments, the derivative excludes a reactive group for coupling to a polymer along which the optical reporter is bound. In some embodiments, the derivative includes a reactive group with a modified reactive moiety and/or a reactive group including a modified linking group connecting the reactive group to the optical reporter.

In embodiments, an optical sensor is configured for the determination of the presence and/or amount of a target in a sample liquid. The optical sensor may be configured in the form of a polymer layer, e.g., a layer of a hydrophilic polymer such as a urethane, e.g., an ether-based hydrophilic urethane. A first surface of the polymer layer is configured to be exposed to a sample fluid, e.g., a sample liquid, containing a target of interest. A second surface of the polymer layer may be secured to a support that is optically transparent at wavelengths useful for exciting and detecting luminescence, fluorescence, from an optical reporter and/or an optical reference present therein. The polymer layer is permeable with respect to the target such that, when the first surface of the polymer layer contacts the sample liquid, target present in the liquid passes through the first surface into the polymer layer, e.g., by diffusion, and interacts with the optical reporter therein. A composition of the polymer layer, e.g., the composition of the polymer of the polymer layer and/or the concentration of one or more of the optical reporter, optical reference, and/or optical isolating reagent may be generally homogeneous proceeding between the first surface configured to be in contact with the sample liquid and the second surface secured to the optical support.

In some embodiments, the optical sensor includes at least one of the foregoing optical reporters or derivative thereof. For example, the optical reporter may be covalently bound to or physically constrained within a polymer layer of the optical sensor. The polymer may be a hydrophilic polymer such as a urethane, e.g., an ether-based hydrophilic urethane.

In some embodiments, the target is an inorganic ion, e.g., Ca⁺⁺, K⁺, Na⁺, or H⁺. In some embodiments, the target is a small molecule, e.g., creatinine, lactate, or glucose. In some embodiments, the target is a protein.

In some embodiments, the optical sensor includes, within a single optical sensing layer, e.g., a single polymer layer, a combination of an optical reporter and an optical isolating reagent, e.g., carbon black. The optical reporter and optical isolating reagent may be distributed generally homogeneously within the optical sensing layer. In some embodiments, the single sensing layer including the optical reporter and optical isolating reagent is the only layer of the optical sensor that participates in the determination of the presence and/or amount of a target in the sample liquid. The single optical sensing layer may have a first surface that contacts the sample liquid and a second surface adhered to an optically transparent support.

The optical sensor may include a light source configured to irradiate the optical sensing layer with excitation light to produce an optical response, e.g., excite fluorescence, from the optical reporter within the optical sensing layer and a detector configured to detect the fluorescence emitted from said excited optical reporter within the optical sensing layer. As used herein, an optical response may include, e.g., a change in fluorescence intensity and/or absorbance. For example, upon binding with or otherwise interacting with a target, the fluorescence intensity of an optical reporter may increase as compared to the fluorescence intensity in the absence of the target.

In some embodiments, a first surface of the optical sensing layer is configured to be exposed to, i.e., be in direct contact with, a sample fluid, e.g., a sample liquid, and an opposed second surface of the optical sensing layer is secured to an optical window through which the light source directs the excitation light and the detector detects fluorescence. In some embodiments, the optical sensing layer is configured to attenuate an average of at least about 50%, at least about 75%, at least about 85%, at least about 90%, at least about 95%, at least about 98%, or at least about 99% of the excitation light impinging upon the second surface of the sensing layer prior to the excitation light reaching the first surface thereof. In some embodiments, the optical sensing layer is configured to attenuate an average of at least about 50%, at least about 75%, at least about 85%, at least about 90%, at least about 95%, at least about 98%, or at least about 99% of light that is emitted at the first surface within a fluorescence wavelength range detected by the detector prior to such light passing through the optical sensing layer and through the second surface thereof. The attenuation of the light may be determined along an optical axis that is normal to the second surface of the optical sensing layer.

In some embodiments, the optical sensing layer has a thickness, in the dry state, of about 250 μm or less, about 125 μm or less, about 75 μm or less, about 50 μm or less, or about 30 μm or less. The optical sensing layer may have a thickness, in the dry state, of at least about 5 μm, at least about 10 μm, at least about 20 μm, or at least about 25 μm. The thickness of the optical sensing layer may be determined along an axis normal to a surface of the optical sensing layer configured to be disposed in contact with a sample liquid. The thickness of the optical sensing layer may change by about 50% or less, about 35% or less, about 25% or less, about 20% or less, or about 15% or less upon exposure to a sample liquid.

In some embodiments, the optical sensor includes an optical medium, e.g., a polymeric medium, that is a mixture including an optical reporter emitting light, e.g., fluorescence, within an emission wavelength band and an optical isolating reagent absorbing light within the emission wavelength band. The optical medium may be configured as a layer, e.g., a polymer layer, with a second surface secured with respect to an optically transmissive support and a first opposing surface accessible to the target or an indicator species indicative of the target present in the sample liquid. A light source is configured to direct excitation light through the optically transmissive support and irradiate the optical reporter residing within the optical medium with the excitation light. A detector is configured to detect light within a detection light band that is emitted by the optical reporter from within the layer and passes through the optically transmissive support. The layer defines a thickness between the first and second surfaces thereof along an axis normal to the second surface. The average transmission over the detection light band of the layer over the thickness is about 50% or less, about 25% or less, about 15% or less, about 10% or less, about 5% or less, about 2% or less, or about 1% or less. The average transmission detection light band of the layer over the thickness may be, e.g., at least about 0.25%, at least about 0.5%, at least about 1%, at least about 2%, or at least about 2.5%. The optical isolating reagent may be carbon black. In some embodiments, the first surface is in direct contact with a surface of the optically transmissive support and the second surface is configured to be placed within direct contact with a sample liquid.

In some embodiments, the optical sensor includes an optical medium that is a mixture including an optical reporter emitting light, e.g., fluorescence, when excited with light having a wavelength within an excitation wavelength band and an optical isolating reagent absorbing light within the excitation wavelength band. The optical medium may be configured as a layer, e.g., a polymer layer, with a second surface secured with respect to an optically transmissive support and a first opposing surface accessible the target or an indicator species indicative of the target present in the sample liquid. A light source is configured to direct excitation light within the excitation wavelength band through the optically transmissive support and irradiate the optical reporter residing within the optical medium with the excitation light. A detector is configured to detect light that is emitted by the optical reporter from within the layer and passes through the optically transmissive support. The layer defines a thickness between the first and second surfaces thereof along an axis normal to the second surface. The average transmission over the excitation light band of the layer over the thickness is about 50% or less, about 25% or less, about 15% or less, about 10% or less, about 5% or less, about 2% or less, or about 1% or less. The average transmission over the excitation light band of the layer over the thickness may be, e.g., at least about 0.25%, at least about 0.5%, at least about 1%, at least about 2%, or at least about 2.5%. The optical isolating reagent may be carbon black. In some embodiments, the first surface is in direct contact with a surface of the optically transmissive support and the second surface is configured to be placed within direct contact with a sample liquid.

In some embodiments, the optical sensor includes an optical medium that is a mixture including an optical reporter emitting light, e.g., fluorescence, within an emission wavelength band when excited with light having a wavelength within an excitation wavelength band and an optical isolating reagent absorbing light within the emission wavelength band and the excitation wavelength band. The optical medium may be configured as a layer, e.g., a polymer layer, with a second surface secured with respect to an optically transmissive support and a first opposing surface accessible to the target or an indicator species indicative of the target present in the sample liquid. A light source is configured to direct excitation light within the excitation wavelength band through the optically transmissive support and irradiate the optical reporter residing within the optical medium with the excitation light. A detector is configured to detect light within a detection light band that is emitted by the optical reporter from within the layer and passes through the optically transmissive support. The layer defines a thickness between the first and second surfaces thereof along an axis normal to the second surface. The average transmission over the excitation wavelength band and the detection light band of the layer over the thickness is about 50% or less, about 25% or less, about 15% or less, about 10% or less, about 5% or less, about 2% or less, or about 1% or less. The optical isolating reagent may be carbon black. The average transmission over the excitation wavelength band and the detection light band of the layer over the thickness may be, e.g., at least about 0.25%, at least about 0.5%, at least about 1%, at least about 2%, or at least about 2.5%. The optical isolating reagent may be carbon black. In some embodiments, the first surface is in direct contact with a surface of the optically transmissive support and the second surface is configured to be placed within direct contact with a sample liquid.

In embodiments, the optical sensor includes an optical sensing layer, e.g., a polymer layer, comprising a mixture of an optical reporter molecule and carbon black, wherein the carbon black is present in an amount of at least about 0.05%, at least about 0.1%, at least about 0.5%, at least about 1%, at least about 2%, at least about 3%, at least about 4%, or at least about 5% by weight of the optical sensing layer. The carbon black may be present in an amount, e.g., of about 5% or less, about 4% or less, about 3% or less, about 2.5% or less, about 2% or less, or about 1% or less by weight of the optical sensing layer. The optical sensor may be configured such that substantially all, essentially all, or the entirety of an optical reporter signal generated by the optical reporter molecule in the optical sensor arises from the optical reporter molecule in the optical sensing layer including the carbon black. The optical sensor may exclude essentially any, e.g., any of the optical reporter that resides apart from the optical sensing layer that also includes the carbon black. In some embodiments, a first surface of the optical sensing layer is configured to be placed in direct contact with a sample liquid and a second opposing surface of the optical sensing layer is in direct contact with a surface of an optically transmissive support through which the optical sensing layer can be irradiated with light to excite fluorescence from the optical reporter and through which fluorescence emitted by the optical reporter from within the optical sensing layer can be detected.

In embodiments, an optical sensor for detecting a target present in a sample liquid includes or optionally consists essentially of a single polymer layer, the polymer layer including an optical reporter and carbon black and, optionally an optical reference, the polymer layer having a first surface configured to contact the sample liquid and a second surface secured to an optically transparent support, wherein the carbon black is present in an amount of at least about 0.1%, 0.2%, 0.3%, 0.4%, 0.5%, 0.6%, 0.7%, 0.8%, 0.9%, or 1% by weight of the single polymer layer. In embodiments, a method of using the optical sensor includes contacting the first surface of the polymer layer with a sample liquid whereupon at least some of the target passes into the polymer layer and interacts with the optical reporter. The polymer layer is irradiated with light through the second surface to excite fluorescence from the optical reporter. The fluorescence is detected and the presence and/or amount of target present in the sample liquid is determined based on the detected fluorescence.

In embodiments, an optical sensor for detecting a target present in a sample liquid includes or optionally consists essentially of a single polymer layer, the polymer layer including an optical reporter and carbon black and, optionally an optical reference, the polymer layer having a first surface configured to contact the sample liquid and a second surface secured to an optically transparent support, wherein the carbon black is present in an amount sufficient to reduce the optical transmittance of light traveling between the first and second surfaces of the polymer layer along an optical axis normal to the second surface to less than about 20%, less than about 15%, less than about 10%, less than about 5%, or less than about 2.5% of the transmittance of the layer in the absence of the carbon black at a wavelength of 475 nm. In embodiments, a method of using the optical sensor includes contacting the first surface of the polymer layer with a sample liquid whereupon at least some of the target passes into the polymer layer and interacts with the optical reporter. The polymer layer is irradiated with light through the second surface to excite fluorescence from the optical reporter. The fluorescence is detected and the presence and/or amount of target present in the sample liquid is determined based on the detected fluorescence.

Any of the optical sensors disclosed herein may be disposed in a detection zone of a microfluidic strip such as a microfluidic strip disclosed in International Application No. PCT/GB2017/051946 filed Jun. 30, 2017 (“the '946 application”) or International Application No. PCT/US2021/013325 filed Jan. 13, 2021 (“the '325 application), which applications are incorporated herein by reference in their entireties. In such embodiments, an internal surface of a wall of the detection zone of the microfluidic strip is an optical support for the optical sensor. In use, the optical sensor is irradiated with excitation light through the wall of the strip and fluorescence generated by the optical reporter and/or optical reference passes through the wall of the strip and is detected. The presence and/or amount of the target is determined based on the detected fluorescence.

In embodiments, a method of forming a polymer including an optical reporter includes combining one or more polymerization monomers and an optical reporter having a polymerization group polymerizable with such monomers to form a polymer having the optical reporter covalently bound therealong. The optical reporter may include, for example, any of the optical reporters disclosed herein. The polymerization group of the optical reporter may include a vinyl group. The monomers may each include a vinyl group. For example, a polymer useful in the manufacture of a sensor directed to determination of the presence and/or amount of Na⁺ may be formed according to the following reaction in the presence of azobisisobutyronitrile (AIBN) and THF:

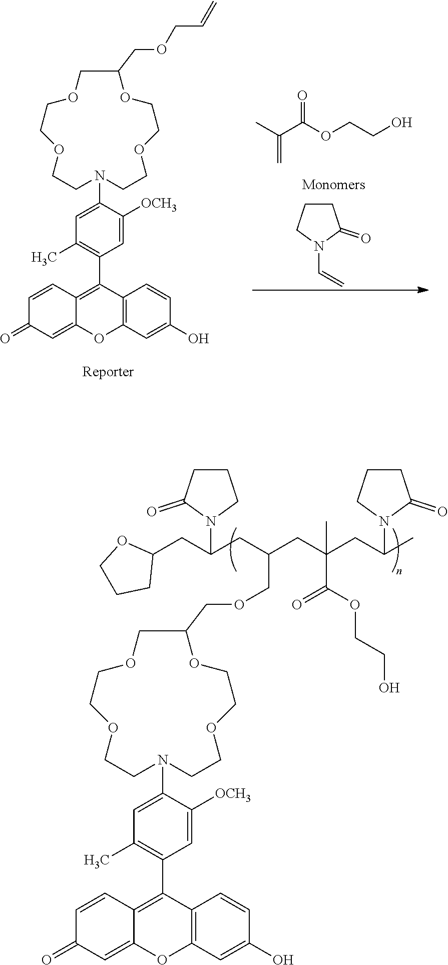

In some embodiments, the method of forming the polymer including an optical reporter further includes combining an optical reference with the monomers and optical reporter such that the formed polymer further includes the optical reporter covalently bound therealong. The optical reference is a compound having a known, e.g., constant, optical response as a function of concentration of the target to which the optical sensor is directed. For example, a polymer useful in the manufacture of a sensor directed to determination of the presence and/or amount of Na⁺ may be formed according to the following reaction in the presence of azobisisobutyronitrile (AIBN) and THF:

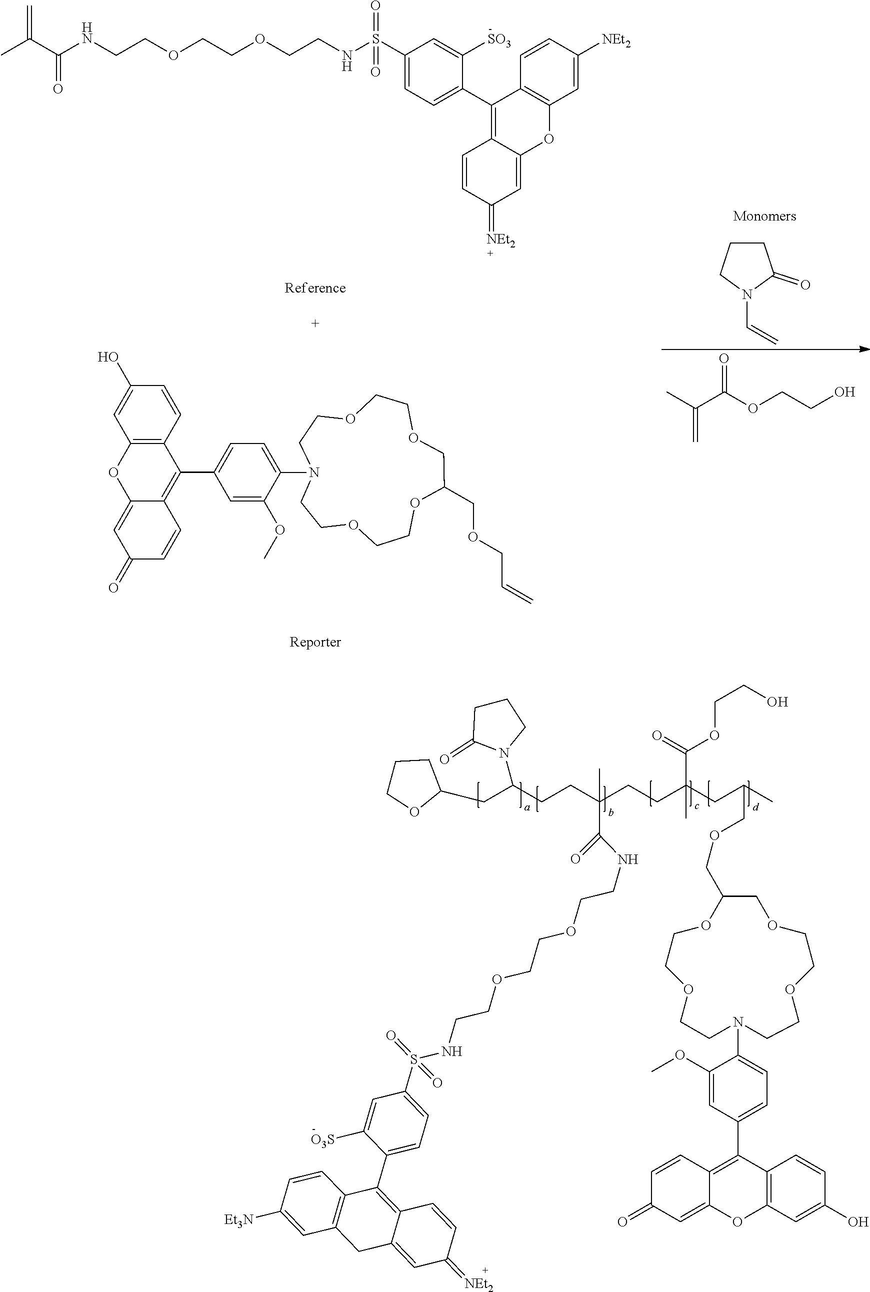

In embodiments, a method of forming a polymer including an optical reference includes combining a one or more polymerization monomers and an optical reporter having a polymerization group polymerizable with such monomers to form a polymer having the optical reference covalently bound therealong. The optical reference is a compound having a known, e.g., constant, optical response as a function of concentration of the target to which the optical sensor is directed. The polymer may exclude an optical reporter. For example, a polymer containing an optical reference may be formed according to the following reaction in the presence of azobisisobutyronitrile (AIBN) and THF:

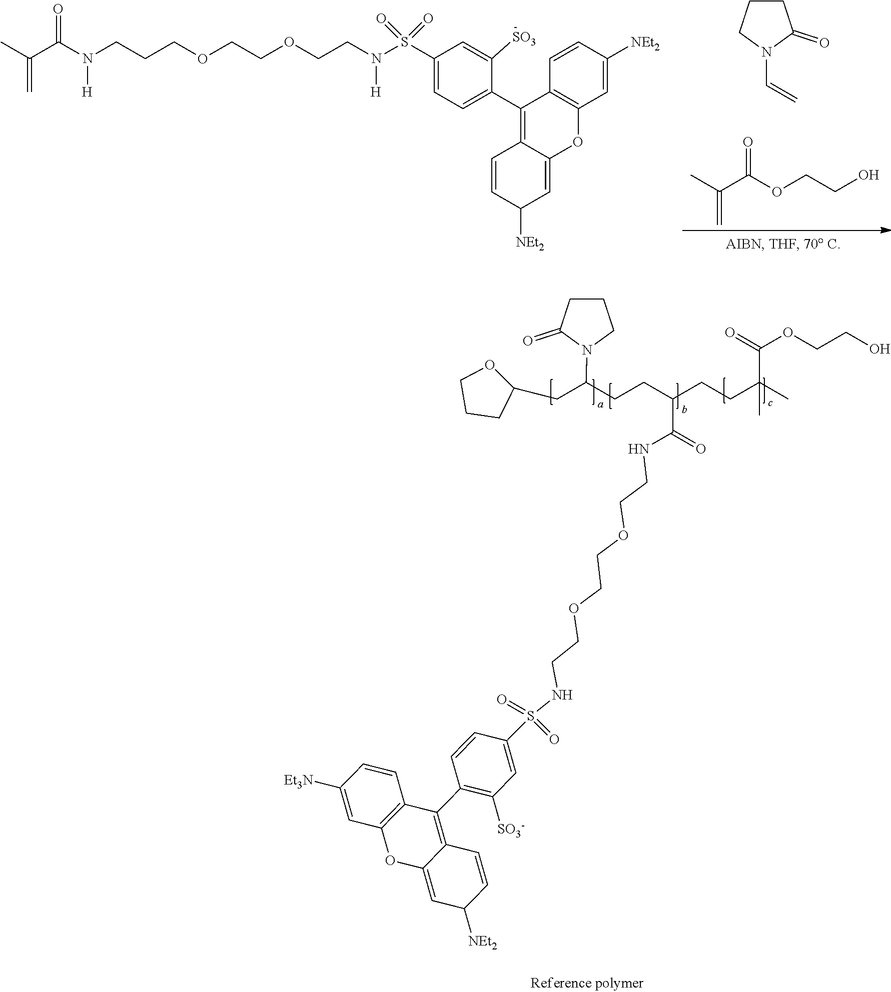

In embodiments, a method of manufacturing an optical sensor medium includes forming a mixture including (i) a hydrophilic polymer; (ii) an optical isolating reagent; (iii) an optical reporter; (iv) an optical reference; and (v) the remainder to 100% solvent, with the percentages being by weight with respect to the total weight of the formed mixture. For example, the formed mixture may include (i) between about 8% to 30% by weight hydrophilic polymer; (ii) between about 0.1% to 5% optical isolating reagent; (iii) between about 5×10⁻⁵% to 8×10⁻²% optical reporter; (iv) between about 0% to 8×10⁻²% optical reference; and (v) the remainder to 100% solvent. A ratio of the weight of optical reporter to the weight of optical reference may be, e.g., at least about 1:1, at least about 2:1, at least about 3:1, or at least about 4:1. The ratio of the weight of optical reporter to the weight of optical reference may be, e.g., about 1:1 or less, about 1:2 or less, about 1:3 or less, or about 1:4 or less.

In some embodiments, the hydrophilic polymer includes a first portion of polymer that is free of optical reporter or optical reference molecules and a second portion of polymer that includes both optical reporter and optical reference molecules covalently bound therealong. For example, the first portion of optical reporter may be between about 0% to 22% and the second portion of polymer may be between about 0.1 and 8% in each case by weight of the total mixture. In some embodiments, the hydrophilic polymer includes a first portion of polymer that is free of optical reporter or optical reference molecules, a second portion of polymer that includes an optical reporter, but no optical reference, covalently bound therealong, and a third portion of polymer that includes an optical reference, but no optical reporter, covalently bound therealong. For example, the first portion of optical reporter may be between about 0% to 22%, the second portion of polymer may be between about 0.1 and 4%, and the third portion of optical reporter may be between about 0.1 and 4%, in each case by weight of the total mixture.

In some embodiments, the optical reporter is covalently bound to at least a portion of the hydrophilic polymer. The mixture may include a first portion of hydrophilic polymer that is free of the optical reporter and a second portion of hydrophilic polymer including, e.g., covalently bound therein or physically entrapped therein, the optical reporter. The first portion of polymer may be between about 0.5% to 15% by weight of the mixture and the second portion of the polymer may be between about 0.5% and 15% by weight of the mixture. The optical reporter may be between about 0.5% to 8% by weight with respect to the total weight of the second portion of polymer.

The optical isolating reagent may be, e.g., a particulate, e.g., carbon black or a carbon nanostructure such as a carbon nanotube and/or a compound such as a molecule having a high optical absorbance over the wavelength range of interest and a low fluorescence, e.g., a low fluorescence dye. The optical isolating reagent may be added to, e.g., at least about 0.05%, at least about 0.1%, at least about 0.25%, at least about 0.5%, at least about 0.75%, at least about 1%, or at least about 2% by weight of the mixture. The optical isolating reagent may be added to, e.g., about 2% or less, about 1% or less, about 0.75% or less, about 0.5% or less, about 0.25% or less, or about 0.1% or less by weight of the mixture. The solvent may include, e.g., a hydrophilic solvent such as water or alcohol such as methanol, ethanol, or isopropyl alcohol, or combination thereof. The method may further include depositing the mixture as a layer on a surface and then drying the mixture. In some embodiments, the optical isolating reagent is present in the dried layer in amount of between about 0.5% to 30% by weight. For example, the optical isolating reagent may be present in the dried layer in amount of at least about 0.5%, at least about 1%, at least about 2.5%, at least about 3.5% or at least about 5% by weight of the dried layer. The optical isolating reagent may be present in the dried layer in an amount of about 30% or less, about 25% or less, about 20% or less, or about 15% or less by weight of the dried layer.

In any of the foregoing embodiments, the optical isolating reagent, e.g., carbon black, may be stabilized. The use of the stabilized optical isolating reagents can improve rheological properties such as viscosity, sedimentation, flow and/or dispensing behavior. For example, the optical isolating reagent may be stabilized using a non-ionic dispersant or slightly anionic dispersant in a concentration of 0.1% to 1% by weight of a polymer solution used to form the optical sensor. Exemplary non-ionic dispersants or slightly anionic dispersants include alcohol alkoxylates such as alcohol ethoxylates, polymeric ethoxylated non-ionic wetting reagents, modified polymers with pigment affinity groups, and/or oxirane, phenyl-, polymer with oxirane, monoalkyl ethers. Exemplary commercial products include Tego® 760W, Tego® 761W, Tego® 755W, and Tego® 650 (Evonik, Essen, Germany). As another example, the optical isolating reagent may be stabilized using a surface modified carbon black that incorporates hydrophilic groups for solvent affinity, with such stabilized carbon black present in an amount of from 0.5% to 1% by weight of a polymer solution used to form the optical sensor. Examples of such dispersants include alcohols, C13-C15, branched and/or linear, ethoxylated. Exemplary commercial products include PX Kappa and OE430W from Orion Engineered Carbons (Sennengerberg, Luxembourg), and XSL from Kremer Pigments (New York, N.Y.). As yet another example, the optical isolating reagent may be stabilized using pre-stabilized carbon black dispersion from 0.5% to 1% by weight of a polymer solution used to form the optical sensor. Exemplary pre-stabilized carbon black dispersions may be stabilized with, e.g., ethylene glycol and surfactants. Suitable commercial products include Aquablack 8367 and Aquablack 8386 from ChromaScape (Independence, Ohio).

In embodiments, an optical sensor is prepared from a mixture including: (i) 2 to 10% by weight hydrophilic urethane polymer; (ii) 0.5% to 1% by weight stabilized optical isolating reagent, e.g., stabilized carbon black; (iii) 5% by weight of any of the foregoing polymers including any of the foregoing optical reporters and, optionally, any of the foregoing optical references; and (iv) the remainder a mixture of water and a water-soluble organic solvent, e.g., a 90:10 EtOH:H2O solution. The optical sensor may be formed by a method including forming a first mixture by combining the optical isolating reagent, e.g., carbon black, and the mixture of water and organic solvent. In the second step, the hydrophilic urethane polymer is added in dry form to the first mixture to form a second mixture. In the third step, the polymer including the optical reporter and optional optical reference is added to the second mixture and mixed to form a third mixture. The third mixture is applied as a thin layer within the detection zone of a microfluidic strip such as a microfluidic strip disclosed in the '946 application or '325 application. The applied mixture is dried for 3 minutes at 65° C., in which time substantially of the solvent solution evaporates and leaves behind an optical sensing layer within the detection zone of the microfluidic strip. An exemplary hydrophilic polymer is HydroMed™ D1, D2, D4, or combination thereof.

In embodiments, a fluorescence calibration device includes a generally planar optically transparent support having opposed first and second surfaces. A fluorescence calibration layer is disposed on the first surface of the support. The fluorescence calibration layer includes at least one fluorescent compound and an optical isolating reagent. The calibration layer has a secured surface disposed in contact with the first surface of the support, e.g., secured with respect thereto, and a second surface opposed to the first surface of the calibration layer along an axis normal to the first surface of the support. The second surface of the support opposing the first surface thereof is disposed outside of the calibration zone, e.g., as an external surface of a microfluidic device containing the calibration layer. For example, the calibration layer and optical support may be respectively arranged and configured as illustrated in FIG. 1 for optical sensing layer 12 and optically transmissive window 20.

The calibration layer may further include a solid or semi-solid matrix in which the fluorescent compound and optical isolating reagent are bound, suspended and/or dissolved. For example, the matrix may be a polymeric matrix, such as any of the permeable or semi-permeable polymers disclosed herein. Alternatively, the matrix may be impermeable to aqueous media, e.g., the matrix may be a polymer generally impermeable to water and/or components of aqueous biological liquids such as buffers, blood, blood plasma, saliva, sputum, nasopharyngeal samples, urine, and combinations of one or more thereof. Such components of biological liquids include such species as ions and biomolecules (e.g., proteins, sugars, and/or lipids). As another example, the matrix may be a polymer generally may be permeable (or impermeable) to water but impermeable to components of aqueous biological samples.

The at least one fluorescent compound emits fluorescence within a wavelength range of a fluorescence detection instrument to be calibrated. For example, the fluorescent compound may have a fluorescence emission maximum within the range of about 400 nm to about 850 nm. Exemplary fluorescent compounds include fluorescent dyes such as rhodamine dyes. In embodiments, the calibration medium includes more than one fluorescent compound, e.g., two fluorescent compounds. Each compound may have a different fluorescence spectrum to permit calibrating an instrument in each of multiple different wavelength regions. In addition, a method of using the calibration medium may include determining a ratio or other measure indicative of the relative fluorescence intensity of the two fluorescent compounds. Such determination permits calibrating the response of a fluorescence instrument in both the intensity domain and the wavelength domain. The optical isolating reagent may be any of the optical isolating reagents disclosed herein, e.g., carbon black, and may be present in an amount by weight disclosed herein for such optical isolating reagents.

The support may be a layer of a diagnostic device that is configured to perform an assay to determine the presence and/or amount of one or more targets in a liquid sample applied to the device. For example, the diagnostic device may be a microfluidic strip such as a microfluidic strip disclosed in the '946 application or '325 application. Suitable diagnostic devices typically include one or more reagents, e.g., reagents configured to bind to and/or detect a target, configured to facilitate the assay for the one or more targets. The calibration layer may be disposed within a dedicated calibration zone of the device, e.g., the first surface of the support may be disposed within such calibration zone. A dedicated calibration zone is a calibration zone which is not used for determining the presence of one or more targets in a sample added to the diagnostic device. Instead, such calibration zone is used solely to obtain a fluorescence signal used to calibrate a fluorescence instrument. Such calibration zone may be disposed within a channel of the diagnostic device that lacks reagents to facilitate the detection of the one or more targets.

Alternatively, the calibration layer may be disposed in a detection zone configured to also perform the detection of the one or more targets present in the detection zone. Such combined calibration zone and detection zone is disposed within a channel of the diagnostic device that may further includes one or more reagents to perform the detection of the one or more targets. The arrangement of the calibration layer with respect to the detection zone may be as described for the calibration layer in a dedicated calibration zone.

The first surface of the transparent support may include an opaque frame within which the calibration layer is disposed. The opaque frame defines the maximum area along (e.g., in the plane of) the first surface of the support from which fluorescence from the calibration layer is detected. For example, the area within the opaque frame may be about 1 mm², about 2 mm², about 3 mm², about 4 mm², or about 5 mm². Such an opaque frame may be formed by, e.g., screen printing an opaque ink onto the first surface of the support.

In use, the calibration layer is irradiated with light by passing light through the optical support and into the calibration layer. Fluorescence emitted by the fluorescent compound within the calibration layer passes out of the calibration layer through the optical support to an optical detector. For example, the irradiation and detection may be performed as illustrated in FIG. 1 with respect to irradiating optical sensor layer 12 and detecting fluorescence emitted therein. The optical isolating reagent attenuates, e.g., absorbs and/or scatters at least some of the excitation light within the calibration layer and/or at least some of the fluorescence emitted by the fluorescent compound. Accordingly, the optical isolating reagent determines the depth along an axis normal to the first surface of the support from which fluorescence from the fluorescent compound within the calibration layer is detected. For example, the optical isolating reagent may be present in an amount sufficient to limit the fluorescence detection to less than about 75%, less than about 65%, less than about 50%, less than about 40%, less than about 30%, less than about 25%, or less than about 20% of the thickness of the calibration layer along the axis normal to the first surface when the secured surface of the calibration layer is irradiated by passing light through the optical support from the second surface to the first surface thereof, e.g., as disclosed in FIG. 1 for irradiating the optical sensor disclosed herein. By “limit the fluorescence detection” it is meant that no more than about 15%, no more than about 10%, no more than about 5%, no more than about 2.5%, no more than about 1% or essentially none of the detected fluorescence arises from locations within the calibration layer disposed beyond the aforementioned thickness limits along the axis normal to the first surface. Alternatively, or in combination, the optical isolating reagent may be present in an amount sufficient to essentially exclude the excitation and detection of fluorescence arising from a medium, e.g., a biological sample, in contact with a surface of the calibration layer that opposes a surface of the calibration layer disposed on the first surface of the transparent support.

In embodiments, the calibration layer has a thickness along an axis normal to the first surface of the optical support of at least about 5 μm, at least about 10 μm, at least about 20 μm, or at least about 30 μm. Such thickness may be, e.g., about 100 μm or less, about 75 μm or less, about 50 μm or less, about 40 μm or less, or about 30 μm or less.

In embodiments, the calibration zone or detection zones includes at least two, e.g., 2, 3 or 4, calibration layers, each having a different concentration of fluorescent compound. In use, the fluorescence from each of plurality of calibration media is used to form a fluorescence intensity calibration curve to increase the precision and accuracy of calibration as compared to calibration performed using a single calibration layer.

In embodiments, a method of using the calibration device includes inserting the calibration device into a fluorescence instrument to be calibrated. When inserted, the calibration layer(s) of the calibration device is/are disposed within optical illumination area of the fluorescence instrument so that the calibration layer may be irradiated with a fluorescence excitation source of the instrument and the resulting fluorescence detected by a fluorescence detector of the instrument. In some embodiments, the calibration may be performed with the calibration layer in a dry state. For example, the calibration may be performed without introducing a liquid sample to the calibration device and/or the calibration may be performed with the calibration layer disposed in a channel in which liquid sample does not enter. In other embodiments, the calibration is performed after adding a non-fluorescent liquid to the calibration device such that the liquid contacts the second surface of the calibration layer disposed therein.

Such non-fluorescent liquid may be, e.g., an aqueous liquid such as water or a buffer. In still other embodiments, the calibration device is a diagnostic device as discussed above and the calibration is performed after adding a sample liquid to diagnostic device. The calibration is performed with at least some of the sample liquid disposed in contact with the second surface of the calibration layer. Prior to performing the calibration, gas pressure may be used to remove at least some of the liquid from the calibration layer, e.g., as disclosed in the'946 application or '325 application.

In embodiments, a method of performing an assay for one or more targets includes contacting a liquid sample suspected of containing the one or more targets with (i) a fluorescent detection reagent configured to emit a fluorescence signal indicative of the presence of a target and (ii) at least one calibration layer including a fluorescent compound and an optical isolating reagent. The fluorescent detection reagent and the calibration layer(s) are irradiated with light within the fluorescence excitation range of the fluorescent detection reagent and fluorescent compound of the calibration layer. The optical isolating reagent attenuates, e.g., absorbs or scatters, at least some of the light entering the calibration layer. Respective fluorescent emissions from the fluorescent detection reagent and the fluorescent compound of the calibration layer(s) are detected separately, e.g., from respective spaced-apart locations. The optical isolating reagent attenuates, e.g., absorbs or scatters, at least some of the fluorescence emitted by the fluorescent compound of the calibration layer before the fluorescence exits the calibration layer. The presence and/or amount of the one or more targets is determined based on the detected fluorescent emissions from the fluorescent detection reagent and the fluorescent compound of the calibration layer(s). For example, the determination may include forming a ratio of the detected fluorescence emissions. Alternatively, or in combination, the determination may include forming a calibration curve including intensities of fluorescence from each of multiple calibration layers and determining the presence and/or amount of the one or more targets based on the detected fluorescence emissions from the fluorescence detection reagent and the fluorescent compound of the multiple calibration layers.

The contacting may be performed within a channel of a microfluidic device, e.g., a detection zone of a microfluidic device. For example, the microfluidic device including channels and detection zone thereof may be any of the microfluidic devices disclosed in the '946 or '325 applications. The calibration layer(s) may be any of the calibration media disclosed herein. For example, the calibration layer may be formed of a polymer, e.g., a semi-permeable or impermeable polymer, and/or include the optical isolating reagent in a composition and concentration as disclosed herein for optical sensor layers.

In embodiments, the fluorescent detection reagent is an optical reporter configured to emit fluorescence that depends on an interaction between the detection reagent and a target. For example, the detection reagent may be any of the optical reporters disclosed herein. The fluorescence detection reagent may be disposed within a polymer layer, e.g., a permeable or semi-permeable polymer layer, as disclosed for any of the optical sensors herein. For example, the fluorescence detection reagent may be covalently bound to the polymer of the polymer medium. The polymer layer within which the detection reagent is disposed is spaced apart from the calibration layer so that the fluorescence from the fluorescence detection reagent and fluorescent compound of the calibration layer can be spatially distinguished, e.g., by detecting the fluorescence with a two-dimensional detector such as a charge-coupled-device.

In embodiments, the fluorescent detection reagent is a detectable label bound to a binding reagent that binds directly or indirectly with a target. For example, the detectable label may be any of the detectable labels disclosed in the '946 application or '325 application. The assay may include forming a complex including (i) the detectable label, (ii) a target, and (iii) a magnetic particle, e.g., as disclosed in the aforementioned applications. A magnetic field generator is used to capture the complex within the channel. The complex is captured at a different location from the calibration layer so that the fluorescence from the fluorescence detection reagent and fluorescent compound of the calibration layer can be spatially distinguished, e.g., by detecting the fluorescence with a two-dimensional detector such as a charge-coupled-device.

### BRIEF DESCRIPTION OF THE FIGURES

FIG. 1 is a side view of an optical sensor of the invention;

FIG. 2 is a graph of fluorescence emitted by a sodium ion optical reporter and an optical reference at different sodium ion concentrations; and

FIG. 3 is a graph of the ratio of the fluorescence of the optical reporter signal S and the optical reference signal S_{ref} obtained from the fluorescence spectra of FIG. 2.

### DETAILED DESCRIPTION

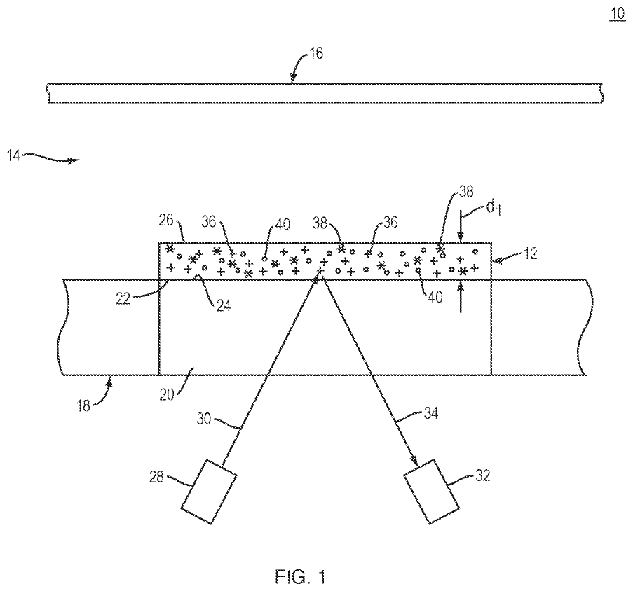

With reference to FIG. 1, an optical sensor 10 is configured to determine the presence and/or amount of a target present in a sample liquid 20. Sensor 10 includes an optical sensing layer 12 disposed within a detection chamber 14 defined by walls 16 and 18. Detection chamber wall 18 includes an optically transmissive window 20. Optical sensing layer 12 has a secured surface 22 secured with respect to an inner window surface 24 of optically transmissive window 20 and an exposed sensor surface 26 exposed to sample liquid within detection chamber 14. Optically transmissive window is an optically transparent support for the optical sensing layer 12.

Optical sensing layer 12 is a semi-permeable polymer matrix and includes, disposed therein, an optical reporter 36, an optical reference 38, and an optical isolating reagent 40. Optical reporter 36 is configured to emit fluorescence when irradiated with excitation light falling within an excitation wavelength band of the optical reporter. Optical reference 38 is configured to emit fluorescence when irradiated with excitation light falling within an excitation wavelength band of the optical reference. The excitation wavelength band of optical reporter 36 and the excitation wavelength band of optical reference 38 may substantially overlap, e.g., be the same, or may have essentially no overlap so that light within the excitation wavelength band of one does not efficiently excite fluorescence from the other. The fluorescence emission range of the optical reporter 36 and optical reference 38 may substantially overlap, e.g., be the same, or may have essentially no overlap so that light detected within a fluorescence emission band of one can be distinguished from light detected within a fluorescence emission band of the other. Typically, the excitation wavelength bands of the optical reporter and optical reference are different and/or the fluorescence emission bands of the optical reporter and optical reference are different so that the reporter and reference responses can be readily distinguished.

Optical sensor 10 also includes a light source 28 and a detector 32. Light source 28 irradiates optical sensing layer 12 through window 20 with excitation light 30 falling within the excitation wavelength band of the optical reporter 36 and falling within the excitation wavelength band of the optical reference 38. Detector 32 detects fluorescence 34 through window 20 emitted from each of the irradiated optical reporter 36 and optical reference 38 residing within optical sensing layer 12. Window 20 acts as a support for optical sensing layer 12 and permits the excitation and detection of luminescence from within optical sensing layer 12, but window 20 does not otherwise participate in the determination of a particular target.

Optical isolating reagent 40 attenuates light at wavelengths falling within the excitation wavelength band(s) and/or the fluorescence emission band(s) of the optical reporter and the optical reference. For example, the average transmittance of sensing layer 12 for light impinging upon sensing layer 12 from light source 28 within the excitation wavelength bands of one or both of optical reporter 36 and optical reference 38 may be about 50% or less, about 25% or less, about 15% or less, about 10% or less, about 5% or less, about 2% or less, or about 1% or less over a distance dl corresponding to the thickness of sensing layer 12. The average transmittance of sensing layer 12 for light impinging upon sensing layer 12 from light source 28 within the excitation wavelength bands of one or both of optical reporter 36 and optical reference 38 may be at least about 0.5%, at least about 1%, at least about 2%, or at least about 3% over a distance dl corresponding to the thickness of sensing layer 12. Alternatively, or in combination, the average transmittance of sensing layer 12 for light detected by detector 32 within the fluorescence emission bands of one or both of optical reporter 36 and optical reference 38 may be about 50% or less, about 25% or less, about 15% or less, about 10% or less, about 5% or less, about 2% or less, or about 1% or less over distance dl corresponding to the thickness of sensing layer 12. The average transmittance of sensing layer 12 for light detected by detector 32 within the fluorescence emission bands of one or both of optical reporter 36 and optical reference 38 may be at least about 0.5%, at least about 1%, at least about 2%, or at least about 3% over a distance dl corresponding to the thickness of sensing layer 12.

Because the optical isolating reagent reduces the amount of excitation light that passes through the optical sensing layer into the sample liquid, the amount of background fluorescence generated within the sample liquid is reduced. In addition, optical isolating reagent attenuates the amount of such background fluorescence within the fluorescence emission bands of the optical reporter and optical reference that passes through the optical sensing layer and reaches the detector. The optical isolating reagent also attenuates some excitation light that would have otherwise reached the optical reporter and optical reference and some of the fluorescence emitted therefrom that would have otherwise reached the detector. However, because the optical sensing layer is semi-permeable with respect to the target, the target diffuses through exposed sensor surface 26 into the optical sensing layer where it reacts with the optical reporter. Therefore, the average pathlength within the optical sensing layer for excitation light reaching the optical reporter and optical reference is shorter than the average pathlength for excitation light passing through the sensing layer into the sample liquid. Therefore, the excitation light is attenuated to a lesser extent before reaching the optical reporter and optical reference than for the sample liquid. The average pathlength within the optical sensing layer for fluorescence emitted by the optical reporter and optical reference is also shorter than the average pathlength for fluorescence emitted within the sample liquid and passing through the optical sensing layer and out of the second surface 22 before being detected by detector 32. Therefore, the fluorescence emitted by the optical reporter and optical reference is attenuated to a lesser extent before reaching the detector than for fluorescence generated within the sample liquid. As a result, the background fluorescence is attenuated to a greater extent than the optical reporter fluorescence so that the signal to noise ratio is increased as compared to the absence of the optical isolating agent, e.g., carbon black.

The optical isolating reagent of the optical sensing layer, e.g., carbon black, may be stabilized. The use of the stabilized optical isolating reagents can improve rheological properties such as viscosity, sedimentation, flow and/or dispensing behavior. For example, the optical isolating reagent may be stabilized using a non-ionic dispersant or slightly anionic dispersant in a concentration of 0.1% to 1% by weight. Exemplary non-ionic dispersants or slightly anionic dispersants include alcohol alkoxylates such as alcohol ethoxylates, polymeric ethoxylated non-ionic wetting reagents, modified polymers with pigment affinity groups, and/or oxirane, phenyl-, polymer with oxirane, monoalkyl ethers. Exemplary commercial products include Tego® 760W, Tego® 761W, Tego® 755W, and Tego® 650. As another example, the optical isolating reagent may be stabilized using a surface modified carbon black from 0.5% to 1% by weight that incorporates hydrophilic groups for solvent affinity. Examples of such dispersants include alcohols, C13-C15, branched and/or linear, ethoxylated. Exemplary commercial products include PX Kappa and OE430W from Orion Engineered Carbons (Sennengerberg, Luxembourg), and XSL from Kremer Pigments (New York, N.Y.). As yet another example, the optical isolating reagent may be stabilized using pre-stabilized carbon black dispersion from 0.5% to 1% by weight. Exemplary pre-stabilized carbon black dispersions may be stabilized with, e.g., ethylene glycol and surfactants. Suitable commercial products include Aquablack 8367 and Aquablack 8386 from ChromaScape (Independence, Ohio).

### EXAMPLES

### Examples 1 and 2 Refer to the Following Diagram

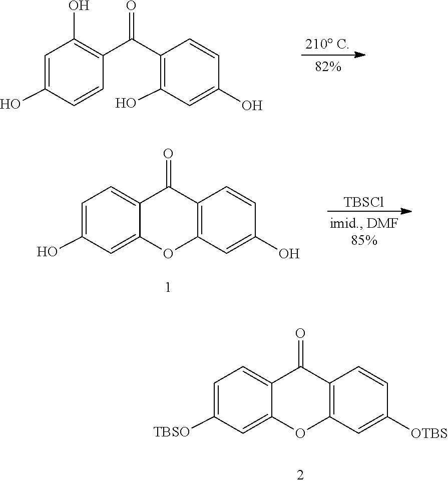 [SMILES: O=C(c1ccc(O)cc1O)c1ccc(O)cc1O ; O=c1c2ccc(O)cc2oc2cc(O)ccc12 ; "TBSCl" ; CC(C)(C)[Si](C)(C)Oc1ccc2c(=O)c3ccc(O[Si](C)(C)C(C)(C)C)cc3oc2c1]

### Example 1: Synthesis of 3,6-dihydroxy-9H-xanthen-9-one (1)

2,2′,4,4′-Tetrahydroxybenzophenone (5.0 g, 20.3 mmol) was dispensed in ten equal portions into glass culture tubes sealed loosely with a rubber septum and heated to 210° C. on a heat block. The reaction liquefied then re-solidified upon heating overnight. The tubes were cooled and the solids suspended in MeOH and combined. The volume was reduced to approximately 30 mL by rotary evaporation and the solids were collected by filtration and rinsed with a little MeOH then dried in vacuo to give (1) (3.78 g, 82%) as a brown solid. mp>250° C. (R_{f}=0.46, 2:8 hexanes:EtOAc). Observed [M+H]⁺=229.0 m/z (calc'd for C₁₃H₉O₄ 229.2 m/z).

### Example 2: Synthesis of 3,6-bis((tert-butyldimethylsilyl)oxy)-9H-xanthen-9-one (2)

3,6-dihydroxy-9H-xanthen-9-one (1) (2.0 g, 8.76 mmol) from Example 1 and imidazole (1.79 g, 26.3 mmol) were dissolved in anhydrous DMF (44 mL) and treated with TBS-Cl (2.91 g, 19.3 mmol) and stirred vigorously at rt. After 4 hrs, the mixture was diluted with EtOAc and washed with 1N HCl (×3), H₂O, brine, dried over Na₂SO₄, and concentrated. The residue was dissolved in CHCl₃ and purified by flash column chromatography on SiO₂ eluted with 95:5 hexanes:EtOAc. The resulting solids triturated with a small volume of hexanes, collected by filtration, and dried in vacuo to give compound 2 (3.42 g, 85%) as a white crystalline solid. mp 152-153° C. (R_{f}=0.43, 9:1 hexanes:EtOAc). ¹HNMR (400 MHz, CDCl₃): δ 0.28 (s, 12H), 1.01 (s, 18H), 6.83 (s, 2H), 6.85 (dd, J=9.2, 2.2 Hz, 2H), 8.20 (ddd, J=9.2, 1.4, 1.4 Hz, 2H). ¹³C NMR (10 MHz, CDCl₃): δ −4.21, 18.42, 25.70, 107.49, 116.57, 117.73, 128.34, 157.89, 161.51, 175.88.

### Examples 3 and 4 Refer to the Following Diagram

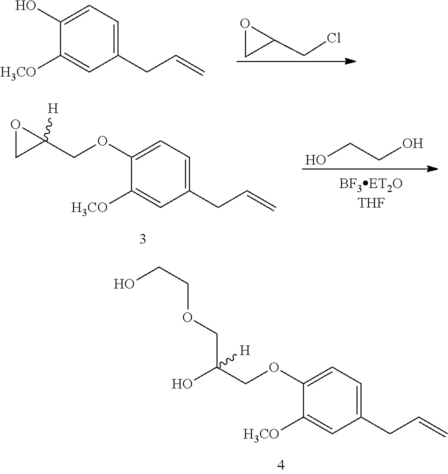

### Example 3: Synthesis of (±)-2-((4-allyl-2-methoxyphenoxy)methyl)oxirane (3)

Eugenol (7.4 g, 45.1 mmol), ±-ephichlorohydrin (10.6 mL, 135 mmol), and Bu₄NHSO₄ (766 mg, 2.26 mmol) were dissolved in 1,4-dioxane (23 mL) and treated with freshly prepared 5M NaOH (27 mL) and heated in a pre-heated 80° C. oil bath for 30 min. The mixture was then cooled on ice and partitioned between EtOAc and sat. NaHCO₃. The aqueous was extracted once with EtOAc and the combined extract was washed with sat. NaHCO₃, brine, dried over Na₂SO₄, and concentrated. The residue was purified by flash column chromatography on SiO₂ eluted with 15→20% EtOAc in hexanes to give (3) (7.5 g, 75%) as a colorless oil. (R_{f}=0.27, 8:2 hexanes:EtOAc). ¹H NMR (400 MHz, CDCl₃): δ 2.73 (dd, J=4.8, 2.7 Hz, 1H), 2.89 (dd, J=4.8, 4.2 Hz, 1H), 3.33 (br d, J=6.7 Hz, 1H), 3.36-3.41 (m, 1H), 3.38 (s, 3H), 4.02 (dd, J=11.4, 5.5 Hz, 1H), 4.21 (dd, J=11.4, 3.6 Hz, 1H), 5.04-5.07 (m, 1H), 5.07-5.11 (m, 1H), 5.95 (dddd, J=16.9, 10.2, 6.7, 6.7 Hz, 1H), 6.69-6.74 (m, 2H), 6.87 (d, J=8.1 Hz, 1H).

### Example 4: Synthesis of (±)-1-(4-allyl-2-methoxyphenoxy)-3-(2-hydroxyethoxy)propan-2-ol (4)

(±)-2-((4-allyl-2-methoxyphenoxy)methyl)oxirane from Example 3 (7.5 g, 34 mmol) was dissolved in anhydrous THE (34 mL) and anhydrous ethylene glycol (34 mL) and cooled to 0° C. then treated with BF₃.Et₂O. The mixture was stirred for 30 min, then the cooling bath was removed and stirring continued for an additional 5 hrs. The mixture was diluted with EtOAc and washed with sat. NaHCO₃(×3), brine (×2), dried over Na₂SO₄, and concentrated. The residue was purified by flash column chromatography on SiO₂ eluted with 1→5% MeOH in CH₂Cl₂ to give (4) (7.47 g, 78%) as a colorless, viscous oil. (R_{f}=0.43, 9:1 hexanes:EtOAc). ¹H NMR (400 MHz, CDCl₃): δ 3.30 (br s, 2H), 3.33 (br d, J=6.68 Hz, 2H), 3.60-3.63 9 m, 2H), 3.65 (d, J=5.8 Hz, 1H), 3.69 (dd, J=10.1, 4.0 Hz, 1H), 3.72-3.76 (m, 1H), 3.84 (s, 3H), 4.02 (dd, J=9.8, 6.5 Hz, 1H), 4.05 (dd, J=9.6, 4.5 Hz, 1H), 4.15-4.22 (m, 1H), 5.04-5.06 (m, 1H), 5.06-5.10 (m, 1H), 5.94 (dddd, J=16.9, 10.2, 6.7, 6.7 Hz, 1H), 6.69-6.73 (m, 2), 6.85 (d, J=8.6 Hz, 1H).

### Examples 5-9 Refer to the Following Diagram

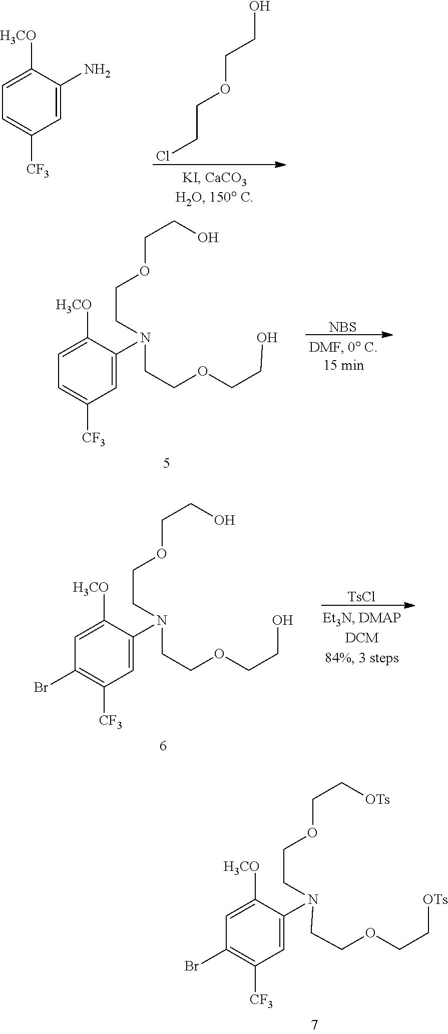

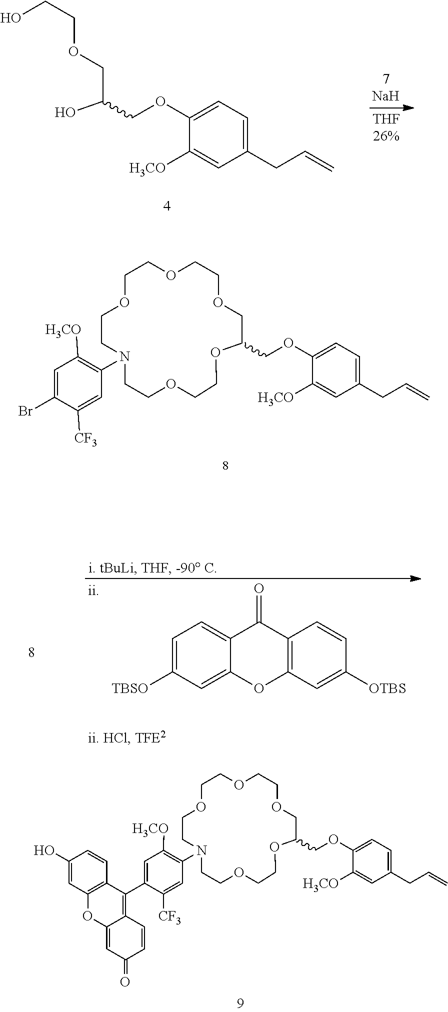 [SMILES: C=CCc1ccc(OCC(O)COCCO)c(OC)c1 ; C=CCc1ccc(OCC2COCCOCCOCCN(c3cc(C(F)(F)F)c(Br)cc3OC)CCOCCO2)c(OC)c1 ; CC(C)(C)[Si](C)(C)Oc1ccc2c(=O)c3ccc(O[Si](C)(C)C(C)(C)C)cc3oc2c1 ; C=CCc1ccc(OCC2COCCOCCOCCN(c3cc(C(F)(F)F)c(-c4c5ccc(=O)cc-5oc5cc(O)ccc45)cc3OC)CCOCCO2)c(OC)c1]

### Example 5: 2,2′-((((2-methoxy-5-(trifluoromethyl)phenyl)azanediyl)bis(ethane-2,1-diyl))bis(oxy))bis(ethan-1-ol) (5)

A 150 mL pressure bottle was charged with 2-methoxy-5-(trifluoromethyl)aniline (972 mg, 5.09 mmol. Caution: stench chemical.), KI (1.27 g, 7.64 mmol), CaCO₃ (560 mg, 5.60 mmol), and 2-(2-chloroethoxy)ethanol (1.27 mL, 7.64 mmol) and heated to 150° C. under N₂ atmosphere overnight. The reaction was cooled, vented, and additional CaCO₃ (1.0 g) and 2-(2-chloroethoxy)ethanol (2 mL) was introduced and the mixture re-heated to 150° C. After 6 hrs, the mixture was cooled and diluted with 1N HCl then washed with Et₂O (×2). The combined wash was back-extracted with 1N HCl once and the combined aqueous was basified with K₂CO₃, shaken with EtOAc, and filtered through Celite. The organic phase was collected and the aqueous extracted twice more with EtOAc. The combined extract was washed with brine, dried over Na₂SO₄, and concentrated to give (5) (2.3 g, >100%) of an orange colored oil which was used without further purification.

### Example 6: 2,2′-((((4-bromo-2-methoxy-5-(trifluoromethyl)phenyl)azanediyl)bis(ethane-2,1-diyl))bis(oxy))bis(ethan-1-ol) (6)

The aniline compound (5) from Example 5 (2.3 g crude, 5.09 mmol) was dissolved in DMF, cooled to 0° C., and treated with solid N-bromosuccinimide (906 mg, 5.09 mmol). After 5 min, additional NBS (75 mg) was added. After an additional 5 min, the reaction mixture was poured into 1N HCl (75 mL) and washed with Et₂O (2×50 mL). The combined wash was back-extracted with 1N HCl (25 mL) and the combined aqueous was neutralized with K₂CO₃ and extracted with EtOAc (×3). The combined extract was washed with brine (×2), dried over Na₂SO₄, and concentrated to give (6) as a brown oil which was used without further purification.

### Example 7: ((((4-bromo-2-methoxy-5-(trifluoromethyl)phenyl)azanediyl)bis(ethane-2,1-diyl))bis(oxy))bis(ethane-2,1-diyl) bis(4-methylbenzenesulfonate) (7)

The diol compound (6) from Example 6 (2.5 g crude, 5.09 mmol) was dissolved in anhydrous DCM (10 mL), cooled to 0° C., and treated with tosyl chloride (2.13 g, 11.2 mmol), Et₃N (1.77 mL, 12.7 mmol), and DMAP (62 mg, 0.51 mmol). The cooling bath was removed and the mixture was stirred at rt for 75 min then diluted with EtOAc and washed with sat. NaHCO₃ (×3), brine, dried over Na₂SO₄, and concentrated. The residue was purified by flash column chromatography on SiO₂ eluted with 20→40% EtOAc in hexanes to give (7) (3.21 g, 84% over 3 steps) as a faintly yellow oil. (R_{f}=0.39, 6:4 hexanes:EtOAc). Observed [M+H]⁺=754.3/756.3 m/z (1:1) (calc'd for C₃₀H₃₆BrF₃NO₉S₂ 754.1 m/z).

### Example 8: 5-((4-allyl-2-methoxyphenoxy)methyl)-16-(4-bromo-2-methoxy-5-(trifluoromethyl)phenyl)-1,4,7,10,13-pentaoxa-16-azacyclooctadecane (8)

The 1,5-diol compound (4) from Example 4 (1.20 g, 4.25 mmol) and the ditosylate compound (7) from Example 7 (3.21 g, 4.25 mmol) were combined and co-evaporated once from anhydrous THF, dried briefly in vacuo, then redissolved in anhydrous THE (10 mL). A 100 mL round bottom flask fitted with a reflux condenser was flame dried then charged with anhydrous THE (30 mL), and NaH (1.02 g, 25.5 mmol) that had been washed twice with hexanes immediately prior. The mixture was heated to reflux and the solution of compounds (4) and (7) was added dropwise over a period of 30 min. After an additional 4 hrs, the mixture was cooled to rt and carefully poured into a mixture of EtOAc and sat. NH₄Cl. The organic phase was collected and the aqueous extracted twice more with EtOAc. The combined extract was dried over MgSO₄, amended with 5% vol MeOH, and filtered through a pad of basic alumina. The filtrate was concentrated and purified by flash column chromatography on SiO₂ eluted with 20-50% EtOAc in hexanes+2% vol Et₃N to give (8) (777 mg, 26%) as a faintly fellow oil. ¹H NMR (400 MHz, CDCl₃): δ 3.32 (br d, J=6.6 Hz, 2H), 3.46 (br dd, J=12.5, 6.1 Hz, 4H), 3.52-3.80 (m, 18H), 3.82 (s, 3H), 3.85 (s, 3H), 3.96-4.04 (m, 1H), 4.04-4.10 (m, 2H), 5.94 (dddd, J=16.8, 10.1, 6.7, 6.7 Hz, 1H), 6.66-6.72 (m, 2H), 6.82-6.89 (m, 1H), 7.05 (s, 1H), 7.32 (s, 1H). ¹³C NMR (101 MHz, CDCl₃): δ 39.9, 52.2, 52.5, 56.06, 56.07, 69.7, 69.8, 69.9, 70.0, 70.3, 70.7, 70.9, 71.10, 71.11, 71.7, 78.0, 109.4, 111.6, 112.7, 114.3, 115.7, 117.6, 118.8, 119.6, 119.7, 120.7, 122.1, 122.4, 124.1, 124.8, 130.7, 133.5, 137.8, 139.1, 146.9, 149.7, 155.0. ¹⁹F NMR (101 MHz, CDCl₃): δ −61.0. Observed [M+H]⁺=692.4/694.4 m/z (1:1) (calc'd for C₃₁H₄₂BrF₃NO₈ 692.2 m/z).

### Example 9: 9-(4-(5-((4-allyl-2-methoxyphenoxy)methyl)-1,4,7,10,13-pentaoxa-16-azacyclooctadecan-16-yl)-5-methoxy-2-(trifluoromethyl)phenyl)-6-hydroxy-3H-xanthen-3-one (9)

The aryl bromide compound (8) of Example 8 (150 mg, 0.217 mmol) was dissolved in MeOH and filtered through a pad of basic alumina and the filtrate was concentrated, co-evaporated twice from 2-Me-THF, and dried thoroughly in vacuo. The residue was redissolved in anhydrous 2-Me-THF (1.1 mL) and cooled in to −116° C. (N₂/EtOH) and tert-butyllithium (285 μL, 0.541 mmol, 1.9M in pentane) was added via the cold sidewall. The pale orange suspension was stirred cold for 10 min. Xanthone 2 (119 mg, 0.26 mmol) was dried by gently warming in vacuo then dissolved in anhydrous 2-Me-THF (1 mL) and added slowly to the aryllithium intermediate via the cold sidewall. A dark red color developed. The mixture was stirred cold for 10 min, then the cooling bath was removed and the mixture was stirred at rt for 10 min which caused the color to fade to orange. A mixture of TFE (1.1 mL) and 2N HCl (1.1 mL) was added which caused the color to dissipate, liberating an intermediate green color, and ultimately a bright red/orange color. The mixture was stirred for 10 min then diluted with 0.5M HCl and washed with Et₂O (×2). The ethereal wash was back-extracted with two small portions of 0.5M HCl and the combined aqueous was neutralized with Na₂CO₃ then back-extracted with EtOAc (×3). The extract was dried over MgSO₄ and concentrated, and the residue was purified by flash column chromatography on SiO₂ eluted with 2→12% MeOH in CH₂Cl₂+2% vol Et₃N to give (9) (59.4 mg, 33%) as an orange film. Observed [M+H]⁺=824.2 m/z (calc'd for C₄₄H₄₉F₃NO₁₁ 824.3 m/z).

### Example 10: Preparation of a Sodium Ion (Na⁺) Sensing Layer

Example 10 refers to the following diagram:

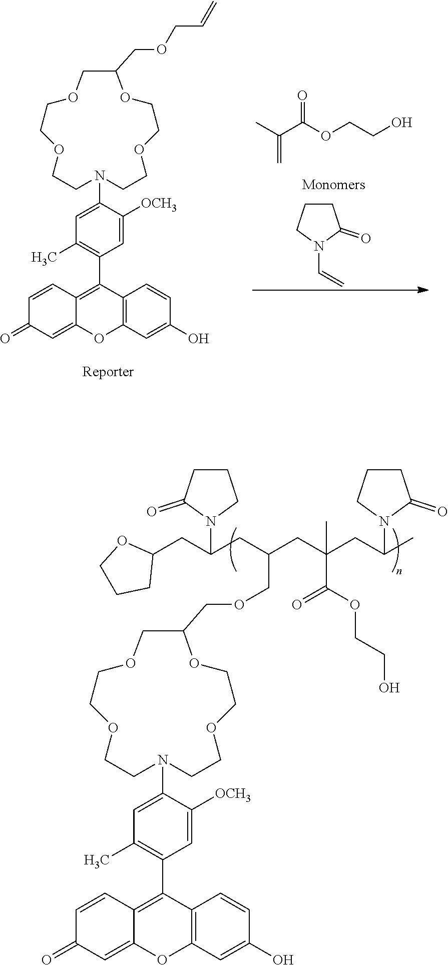

An optical reporter having a terminal vinyl group is reacted with two vinyl monomers in a solution of 2% w/v azobisisobutyronitrile (AIBN) in THE at 70° C. The resulting hydrophilic polymer between about 0.1% to 1% by weight optical reporter, which is covalently bound to the polymer. The polymer is semipermeable with respect to sodium ion.

### Example 11: Preparation of an Optical Sensor for the Determination of Sodium Ion

A mixture is prepared containing: (i) 10% by weight hydrophilic urethane polymer; (ii) 1% by weight carbon black; (iii) 5% by weight of the polymer of Example 10; and (iv) the remainder a 90:10 EtOH:H2O solution. The polymer is applied as a thin layer within the detection zone of a microfluidic strip such as a microfluidic strip disclosed in the '946 application or '325 application. The applied polymer is dried for 3 minutes at 65° C., in which time substantially of the solvent solution evaporates and leaves behind an optical sensing layer within the detection zone of the microfluidic strip.

In use, the microfluidic strip is inserted into a reader, such as one described in the '946 application or '325 application. A liquid sample, e.g., of blood, is applied to the strip. The reader operates the strip to introduce the sample into the detection zone whereupon sodium ion present in the sample diffuses into the optical sensing layer therein and interacts with the optical reporter residing within the layer. The reader has a light source that irradiates the optical sensing layer with excitation light causing the optical reporter to emit fluorescence indicative of the presence and/or amount of sodium ion present in the liquid sample. The carbon black attenuates the excitation light passing through the optical sensing layer into the sample liquid within the detection zone thereby reducing the amount of background fluorescence generated from the sample liquid. In addition, the carbon black attenuates the amount of background fluorescence that is generated before it passes through the optical sensing layer and reaches the detector of the reader. The carbon black also attenuates some excitation light before it reaches the optical reporter and some of the fluorescence emitted by the optical reporter. However, because the sodium ion diffuses into the polymer layer, the average pathlength within the optical sensing layer of such excitation light and fluorescence is less than average pathlength for light traveling through the optical sensing layer into the liquid and for background fluorescence emitted within the liquid and traveling through the optical sensing layer out of the detection zone. Therefore, the background fluorescence is attenuated to a greater extent than the optical reporter fluorescence so that the signal to noise ratio is increased as compared to in the absence of the carbon black.

### Example 12: Preparation of a Sodium Ion (Na⁺) Sensing Layer Including an Optical Reporter and an Optical Reference

Example 12 refers to the following diagram:

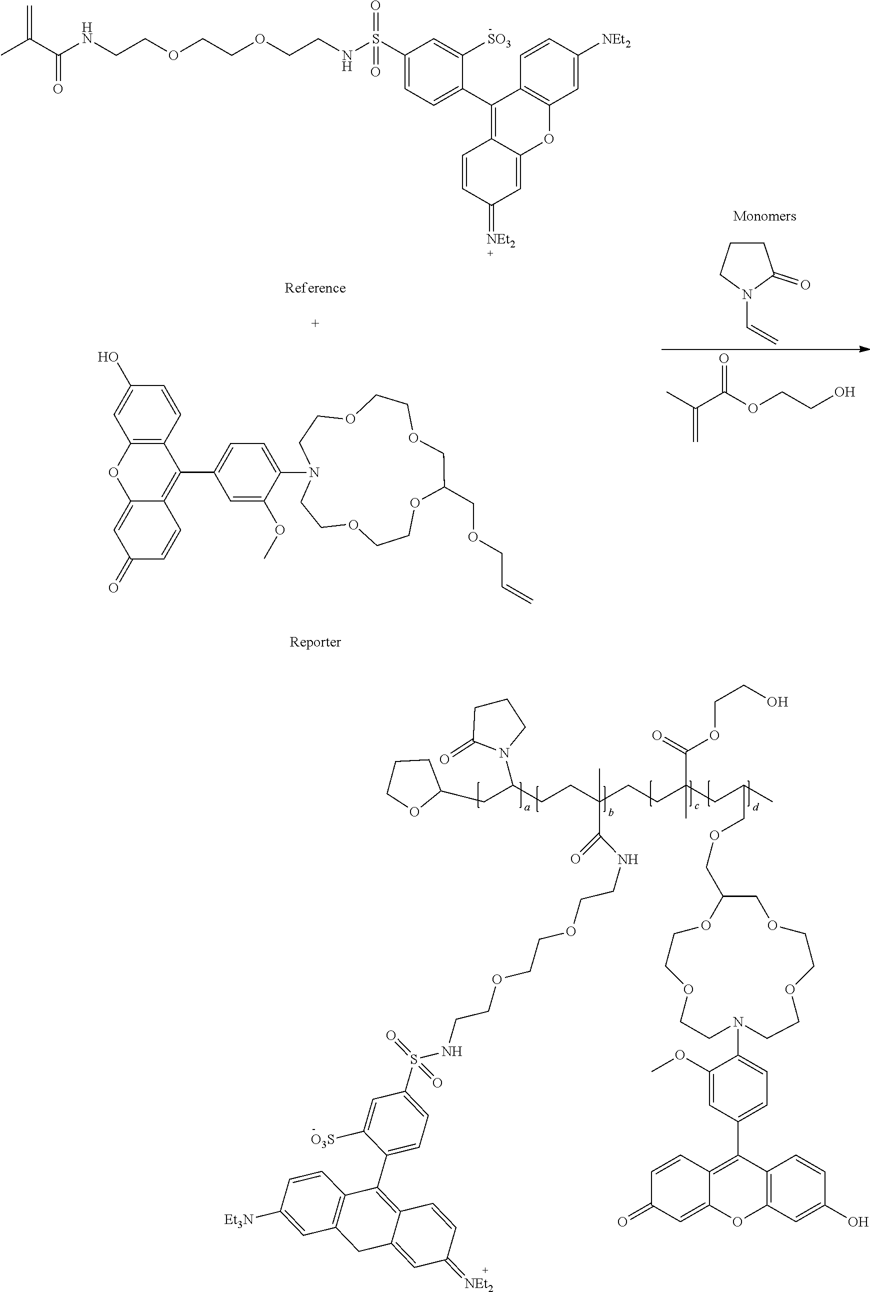

An optical reporter and an optical reference each having a terminal vinyl group are reacted with two vinyl monomers in a solution of 2% w/v azobisisobutyronitrile (AIBN) in THE at 70° C. The resulting hydrophilic polymer between about 0.1% to 1% by weight each of the optical reporter and optical reference, which are covalently bound to the polymer. The polymer is semipermeable with respect to sodium ion.

### Example 13: Preparation of an Optical Sensor with Internal Reference for the Determination of Sodium Ion

A mixture is prepared containing: (i) 10% by weight hydrophilic urethane polymer; (ii) 1% by weight carbon black; (iii) 5% by weight of the polymer of Example 12; and (iv) the remainder a 90:10 EtOH:H2O solution. The polymer is applied as a thin layer within the detection zone of a microfluidic strip such as a microfluidic strip disclosed in the '946 application or '325 application. The applied polymer is dried for 3 minutes at 65° C., in which time substantially of the solvent solution evaporates and leaves behind an optical sensing layer within the detection zone of the microfluidic strip.

### Example 14: Calibration Spectra and Calibration Curve for a Sodium Sensor

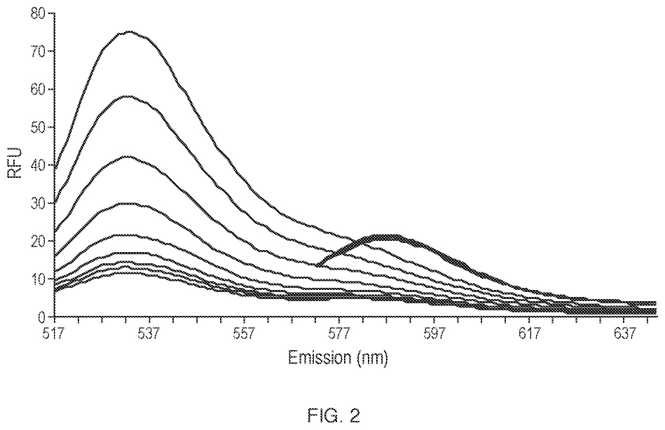

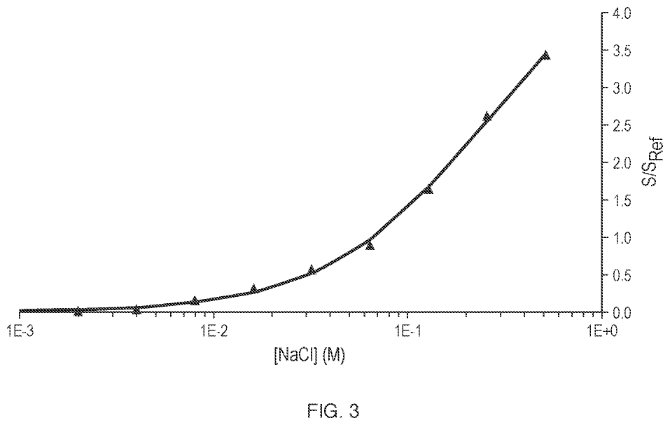

The mixture of Example 13 was applied to a surface of an optical flow cell and dried as per Example 13. The flow cell was inserted into a fluorimeter configure to irradiate the dried sensing layer with excitation light at 490 nm to excite fluorescence from the optical reporter and with excitation light at 550 nm to excite fluorescence from the optical reference. The fluorescence with each excitation light was measured upon exposing the sensing layer to solutions containing different sodium ion concentrations (FIG. 2). The optical reporter emits a fluorescence signal “S” indicative of the presence and/or amount of sodium ion present in the liquid sample. The excitation light causes the optical reference to emit a fluorescence signal “S_{Ref}” the intensity and amount of which is insensitive to the presence and/or amount of sodium ion or concomitant species present in the sample liquid. Accordingly, the optical reporter fluorescence can be normalized by the optical reference fluorescence (S/S_{ref}) to correct for variations in the system thereby increasing the accuracy and precision of the sodium ion determination (FIG. 3). The carbon black attenuates the signals, including the background fluorescence and optical reference and optical reporter fluorescence increasing the signal to noise ratio.

### Examples 15 to 25 Refer to the Following Diagram

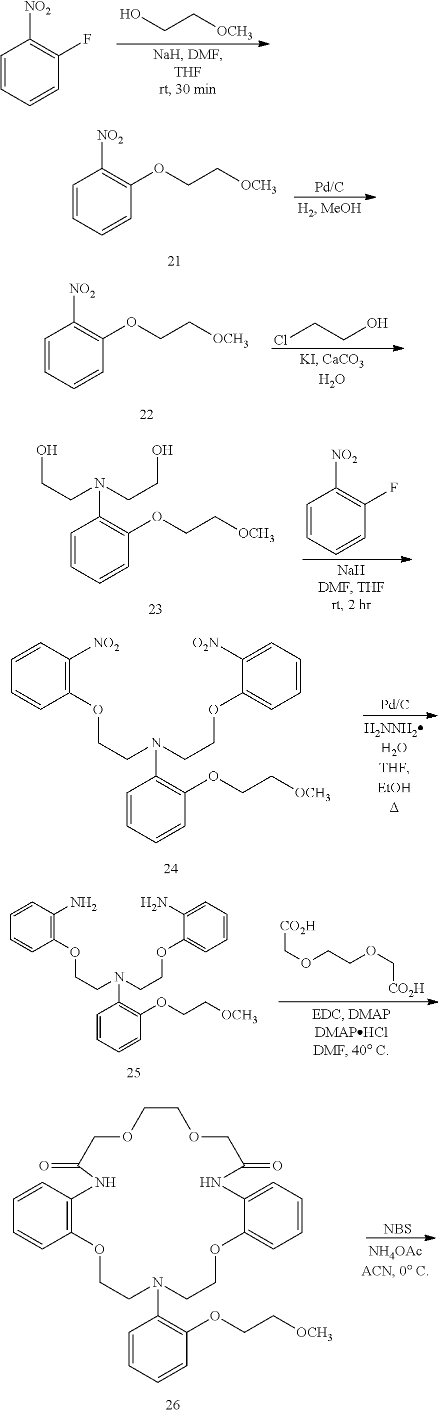

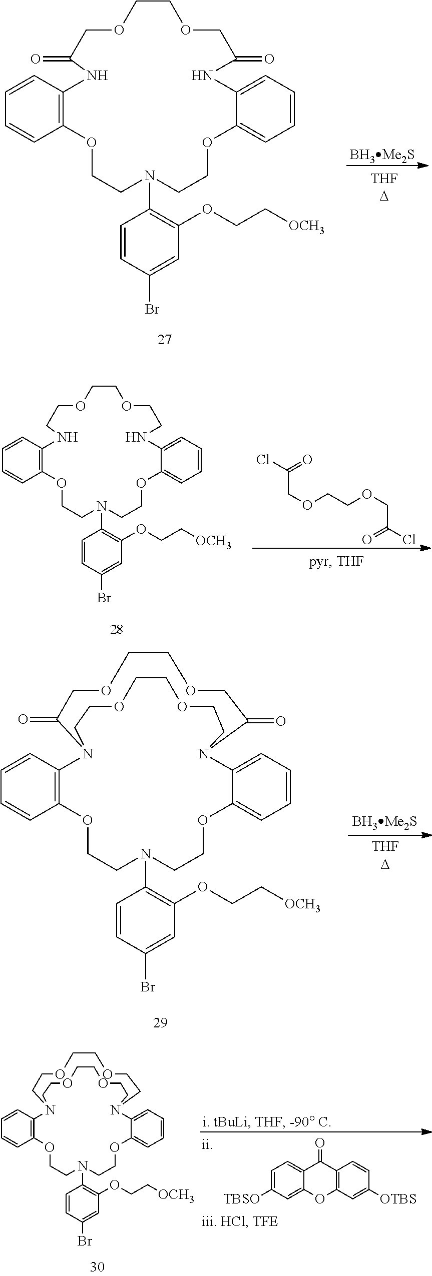

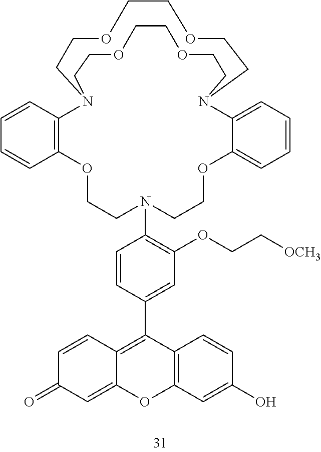 [SMILES: COCCOc1cc(-c2c3ccc(=O)cc-3oc3cc(O)ccc23)ccc1N1CCOc2ccccc2N2CCOCCOCCN(CCOCCOCC2)c2ccccc2OCC1]

### Example 15: 1-(2-methoxyethoxy)-2-nitrobenzene (21)

1-Fluoro-2-nitrobenzene (10 g, 71 mmol) and 2-methoxyethanol (6.5 g, 85 mmol) were dissolved in DMF:THF (30:1, 70 mL) and NaH dispersion (3.4 g, 85 mmol) was added portionwise over approximately 15 min. After an additional 15 min, the reaction was quenched by careful addition of H₂O then diluted with EtOAc and washed with H₂O (×1), K₂CO₃ (×2), brine, dried over Na₂SO₄, and concentrated. The residue was taken up in MeOH and washed with three small portions of hexanes. The methanolic phase was concentrated and distilled in vacuo to give compound (21) (11.1 g, 79%) as an orange oil. Bp 120-130° C., 1 torr. (R_{f}=0.15, 8:2 hexanes:EtOAc). ¹H NMR (400 MHz, CDCl₃): δ 3.45 (s, 3H), 3.78-3.82 (m, 2H), 4.23-4.28 (m, 2H), 7.04 (ddd, J=8.1, 7.4, 1.0 Hz, 1H), 7.11 (dd, J=8.5, 1.0 Hz, 1H), 7.52 (ddd, J=8.5, 7.4, 1.7 Hz, 1H), 7.83 (dd, J=8.1, 1.7 Hz, 1H).

### Example 16: 2-(2-methoxyethoxy)aniline (22)

Nitro compound 21 (11.1 g, 56.1 mmol) was dissolved in MeOH (140 mL). A slurry of Pd/C in MeOH was prepared under N₂ atmosphere and added to the substrate solution. The vessel was evacuated and backfilled with H₂ several times then stirred vigorously for 21 hrs. The mixture was filtered through Celite and the cake carefully rinsed with MeOH. The filtrate was concentrated and purified by flash column chromatography on SiO₂ eluted with 20→40% EtOAc in hexanes to give compound (22) (8.48 g, 90%) as a pale brown oil. (R_{f}=0.38, 6:4 hexanes:EtOAc). ¹H NMR (400 MHz, CDCl₃): δ 3.45 (s, 3H), 3.74-3.78 (m, 2H), 3.86 (br s, 2H), 4.12-4.17 (m, 2H), 6.69-6.77 (m, 2H), 6.79-6.84 (m, 2H). Observed [M+H]⁺=168.3 m/z (calc'd for C₉H₁₄NO₂ 168.1 m/z).

### Example 17: 2,2′-((2-(2-methoxyethoxy)phenyl)azanediyl)bis(ethan-1-ol) (23)

A 150 mL pressure bottle was charged with aniline 22 (8.48 g, 50.7 mmol), KI (8.42 g, 50.7 mmol), CaCO₃ (45.6 mmol), and 2-chloroethanol (10.2 mL, 152 mmol) and the apparatus was purged with N₂, sealed, and heated to 110° C. for 20 hr. The mixture was diluted with 1N HCl and washed with two small volumes of Et₂₀. The combined wash was back-extracted with 1N HCl once. The combined aqueous phase was basified with conc. NH₄₀H at 0° C. and extracted with CH₂Cl₂ (×5). The combined extract was washed with 1M NaOH, brine, dried over Na₂SO₄, then diluted with 0.1 volumes of MeOH filtered through a thin pad of SiO₂ rinsing with 9:1 CH₂Cl₂:MeOH. The filtrate was concentrated to give compound (23) (12.3 g, 95%) as a pale brown oil. ¹H NMR (400 MHz, CDCl₃): δ 3.14-3.18 (m, 4H), 3.44 (s, 3H), 3.45-3.51 (m, 4H), 3.61 (br s, 2H), 3.74-3.78 (m, 2H), 4.11-4.15 (m, 2H), 6.92 (dd, J=8.2, 1.4 Hz, 1H), 6.99 (ddd, J=7.6, 7.6, 1.4 Hz, 1H), 7.13 (ddd, J=8.1, 7.5, 1.7 Hz, 1H), 7.22 (dd, J=7.8, 1.7 Hz, 1H). Observed [M+H]*=256.2 m/z (calc'd for C₁₃H₂₂NO₄ 256.1 m/z).

### Example 18: 2-(2-methoxyethoxy)-N,N-bis(2-(2-nitrophenoxy)ethyl)aniline (24)

Compound 23 (5.49 g, 21.5 mmol) and 1-fluoro-2-nitrobenzene were dissolved in anhydrous DMF:THF (30:1, 27 mL) and NaH dispersion (1.89 g, 47.3 mmol) was added portionwise over a period of approximately 15 min. An exotherm resulted which was not mitigated. After 2 hrs, the reaction was carefully quenched with H₂O then partitioned between EtOAc and H₂O and the aqueous phase extracted twice more with EtOAc. The combined extract was washed with 5% K₂CO₃ (×3), brine, dried over Na₂SO₄, and concentrated. The residue was purified by flash column chromatography on SiO₂ eluted with 30→40% EtOAc in hexanes to give compound (24) (9.9 g, 92%) as a yellow colored oil that crystallized upon standing. Observed [M+H]⁺=498.2 m/z (calc'd for C₂₅H₂₈N₃O₈ 498.2 m/z).

### Example 19: N,N-bis(2-(2-aminophenoxy)ethyl)-2-(2-methoxyethoxy)aniline (25)

bis-Nitro compound 24 (4.98 g, 10 mmol) was dissolved in THF:EtOH (1:1, 50 mL). A slurry of Pd/C in EtOH was prepared under N₂ atmosphere and added to the substrate solution. The mixture was warmed to 50° C. and hydrazine hydrate 4.9 mL was added dropwise, addition of which initially caused effervescence. Following addition, the mixture was warmed to gentle reflux for 12 hrs then cooled to rt and filtered through Celite carefully rinsing with THF. The filtrate was concentrated and the residue was treated with 5% Na₂CO₃ (50 mL) and extracted with EtOAc (3×30 mL). The combined extract was washed with brine, dried over Na₂SO₄, and concentrated. The residue was purified by flash column chromatography on SiO₂ eluted with 50% EtOAc in hexanes to give compound (25) (3.35 g, 76%) as a faintly brown colored viscous oil. (R_{f}=0.31, 1:1 hexanes:EtOAc). ¹H NMR (400 MHz, CDCl₃): δ 3.39 (s, 3H), 3.70-3.76 (m, 6H), 4.09-4.16 (m, 6H), 6.63-6.70 (m, 4H), 6.72-6.80 (m, 4H), 6.87-6.92 (m, 2H), 6.94-7.00 (m, 1H), 7.08-7.12 (m, 1H). Observed [M+H]⁺=438.2 m/z (calc'd for C₂₅H₃₂N₃O₄ 438.2 m/z).

### Example 20: 22-(2-(2-methoxyethoxy)phenyl)-9,10,21,22,23,24-hexahydro-5H,12H,20H-dibenzo[h,q][1,4,10,16]tetraoxa[7,13,19]triazacyclohenicosine-6,13(7H,14H)-dione (26)

bis-Aniline compound 25 (3.35 g, 7.66 mmol) and 3,6-dioxa-1,8-dioic acid were dissolved in anhydrous DMF (20 mL) and added via syringe pump over a period of 90 min to a stirred suspension of EDC.HCl (7.34 g, 38.3 mmol), DMAP (936 mg, 7.66 mmol), and KBF₄ (964 mg, 7.66 mmol) in anhydrous DMF (130 mL) at 40° C. The mixture was stirred for 12 hr then concentrated to approximately one third the initial volume and partitioned between EtOAc and half-saturated NaHCO₃. The aqueous phase was extracted with EtOAc and the combined extract was washed with sat. NaHCO₃ (×2), brine, dried over Na₂SO₄, and concentrated. The residue was purified by flash column chromatography on SiO₂ eluted with 1→3% MeOH in CH₂Cl₂ to give compound (26) (2.51 g, 57%) as a pale yellow oil that solidified upon standing. (R_{f}=0.58, 94:6 CHCl₃:MeOH). ¹H NMR (400 MHz, CDCl₃): δ 3.41 (s, 3H), 3.70-3.74 (m, 2H), 3.84 (t, J=5.7 Hz, 4H), 3.93 (s, 4H), 4.09 (t, J=5.7 Hz, 4H), 4.08-4.12 (m, 2H), 4.15 (s, 4H), 6.75 (d, J=7.8 Hz, 1H), 6.76 (d, J=7.9 Hz, 1H), 6.86-6.91 (m, 2H), 6.91-7.01 (m, 5H), 7.03-7.07 (m, 1H), 8.35 (dd, J=7.6, 1.8 Hz, 1H), 9.20 (br s, 1H). Observed [M+H]⁺=580.3 m/z (calc'd for C₃₁H₃₈N₃O₈ 580.3 m/z).

### Example 21: 22-(4-bromo-2-(2-methoxyethoxy)phenyl)-9,10,21,22,23,24-hexahydro-5H,12H,20H-dibenzo[h,q][1,4,10,16]tetraoxa[7,13,19]triazacyclohenicosine-6,13(7H,14H)-dione (27)

Aniline compound 26 (2.47 g, 3.75 mmol) was dissolved in DMF (21 mL) and treated with NBS (741 mg, 4.16 mmol) with vigorous stirring at rt. After 10 min, additional NBS (82 mg, 463 μmol) was added. After an additional 10 min, the reaction mixture was diluted with EtOAc and washed with Na₂CO₃ (×2), brine, dried over Na₂SO₄, and concentrated. The residue was purified by flash column chromatography on SiO₂ eluted with 0.5→3% MeOH in CH₂Cl₂ to give compound 27 (2.97 g, greater than theory) as a brown oil which crystallized upon standing. (R_{f}=0.37, 99:1 CHCl₃:MeOH). Observed [M+H]⁺=658.4/660.4 m/z (1:1) (calc'd for C₃₁H₃₇BrN₃O₈ 658.2 m/z).

### Example 22: 22-(4-bromo-2-(2-methoxyethoxy)phenyl)-6,7,9,10,13,14,21,22,23,24-decahydro-5H,12H,20H-dibenzo[h,q][1,4,10,16]tetraoxa[7,13,19]triazacyclohenicosine (28)

bis-Amide compound 27 (2.97 g, approx. 4.26 mmol) was dissolved in anhydrous THE (43 mL) and treated with BH₃.Me₂S and the mixture was heated to gentle reflux under N₂ atmosphere for 14 hrs. The mixture was then cooled and quenched by dropwise addition of 3N HCl (21 mL) then returned to reflux for 30 min and again cooled. The solution was rendered basic by the addition of 10M NaOH and extracted with EtOAc (×3). The combined extract was washed with brine (×2), dried over Na₂SO₄, and concentrated. The residue was purified by flash column chromatography on SiO₂ eluted with 30→50% EtOAc in hexanes to give compound 28 (2.33 g, 87%) as a faintly yellow heavy oil. (R_{f}=0.21, 7:3 hexanes:EtOAc). Observed [M+H]⁺=630.2/632.3 m/z (1:1) (calc'd for C₃₁H₄₁BrN₃O₆ 630.2 m/z).

### Example 23: Dioxo-2.2.3-cryptand (29)

bis-Aniline compound 28 (2.33 g, 3.69 mmol) and anhydrous pyridine (654 μL, 8.12 mmol) were dissolved in anhydrous DCM (10 mL). Separately, 3,6-dioxaoctan-1,8-dioic acid chloride (873 mg, 4.06 mmol) was dissolved in anhydrous DCM (10 mL). These two solutions were added via syringe to a 250 mL round bottom flask containing anhydrous THF (74 ml) over a period of approximately 6 hours. The turbid mixture was stirred at rt overnight then diluted with EtOAc and washed with sat NaHCO₃ (×3), brine, dried over MgSO₄, and concentrated. The residue was purified by flash chromatography on SiO₂ eluted with 0→1% MeOH in CH₂Cl₂ with 1% Et₃N to give compound 29 (1.2 g, 42%) as a white foam. Observed [M+H]⁺=772.3/774.3 m/z (1:1) (calc'd for C₃₇H₄₇BrN₃O₁₀ 772.2 m/z).

### Example 24: 2.2.3-cryptand (30)

Diamide compound 29 (1.2 g, 1.55 mmol) was dissolved in anhydrous THE (16 mL) and BH₃.Me₂S (15.5 mmol, 1.47 mL) was added via syringe. The mixture was warmed to 60° C. and stirred for 2 hours. A white precipitate was observed after 1 hour. The reaction was cooled to 0° C. and carefully quenched by dropwise addition of 1N HCl (25 mL) then re-heated to 60° C. for 30 min. The reaction was again cooled to 0° C. and the pH adjusted above 10 by addition of solid NaOH. The mixture was extracted with CHCl₃ (×3), and the combined extract was washed with brine, dried over Na₂SO4 and concentrated. The residue was dissolved in DCM and purified by flash chromatography on SiO₂ eluted with 0→1% MeOH in DCM and 1% Et₃N to give 30 (1.1 g, 95%) as a foam. Observed [M+H]⁺=743.9/746.0 m/z (1:1) (calc'd for C₃₇H₅₁BrN₃O₈ 743.3 m/z).

### Example 25: 2.2.3-Cryptand K⁺ Sensor B (31)

Bromide compound 30 (0.208 mmol, 155 mg) was dissolved in DCM:MeOH (9:1) and filtered through a pipet column of basic alumina then concentrated and further dried in vacuo. The residue was co-evaporated twice from anhydrous 2-methyl-THF then dried in vacuo. The residue was redissolved in anhydrous 2-Me-THF (1.0 mL) and cooled in to −116° C. (N₂/EtOH) and tert-butyllithium (275p L, 0.52 mmol, 1.9M in pentane) was added via the cold sidewall. The suspension was stirred cold for 10 min. Xanthone (116 mg, 0.250 mmol) was dried by gently warming in vacuo then dissolved in anhydrous 2-Me-THF (1 mL) and added slowly to the aryllithium intermediate via the cold sidewall. A dark red color developed. The mixture was stirred cold for 15 min, then the cooling bath was removed and the mixture was stirred at rt for 20 min. A mixture of TFE (1 mL) and 2N HCl (1 mL) was added. The mixture was stirred for 10 min then diluted with 0.5M HCl and washed with 1:1 EtOAc:Hexanes. The aqueous was adjusted to pH 4 and back-extracted with EtOAc (×5). The extract was purified by prep HPCL (10→100% ACN+0.1% HCO₂H over 40 min, 20 ml/min, Clipeus C18 20×250 mm, 10 μm) to give compound 31 (88 mg, 48%) as a green film. Observed [M+H]⁺=876.6 m/z (calc'd for C₅₀H₅₈N₃O₁₁ 876.4 m/z).

### Example 26: Preparation of a Potassium Sensor with Internal Reference

The monomers HEMA and AMP were purified by passing through alkaline aluminum oxide (Acros basic, Brockmann I, for chromatography, 50-200 μm, 60 A, ACROS Organics) to remove inhibitor. The ratio between the monomer to Al₂O₃ was 10 mL/1 g. The purified monomers were kept at 4° C. for short time use (within one week), or −20° C. for long term use. Prior to polymerization, the monomers were brought to room temperature.

A first block polymer of poly(HEMA) containing a reference dye was produced as follows. To a 20 mL vial, were added: an internal reference dye, Rhodamine 594 methacrylate monomer (click chemistry tools, MW: 646.76, 3.00 μmol, 1.94 mg), (E)-2,2′-(diazene-1,2-diyl)bis(2-methylpropanenitrile) (AIBN, MW: 164.21, 3.9 mg, 0.024 mmol), 2-cyanobutan-2-yl-4-chloro-3,5-dimethyl-1H-pyrazole-1-carbodithioate (chain Transfer Reagent MW: 0.144 mmol, 41.4 mg), 2-hydroxyethyl methacrylate (sigma: 128635-500 g; MW: 130.14, 6.00 mmol, 781 mg) and N, N-dimethylformamide (DMF) (3 mL). A magnetic stir bar was added and the vial sealed with a rubber cap. The sealed vial was purged with argon gas. The vial was heated to 70° C. and stirred for 20 hrs at 400 rpm. This formed the first block of poly(HEMA) containing the internal reference rhodamine 594. The vial was cooled down to room temperature.

A second block containing AMP and an Oregon Green® dye was synthesized as follows. To the first block of poly(HEMA) was added: AIBN (MW: 164.21, 3.9 mg, 0.024 mmol), 2-(2,7-difluoro-6-hydroxy-3-oxo-3H-xanthen-9-yl)-5-((2-(methacryloyloxy)ethyl)carbamoyl)benzoic acid monomer (MW: 523.44; 0.017 μmol, 52.0 μg, stock solution 10 μL), N-acryloylmorpholine (AMP) (Sigma-Aldrich® catalog no. 448273-250 mL, 9.0 mmol, 1.27 g), DMF (2 mL). The vial was resealed, purged with argon for 30 minutes. The mixture was heated to 70° C. and stirred for 20 hrs at 400 rpm.

After cooling the mixture to room temperature, 16 mL methanol was added to the flask. The polymer was precipitated by adding 250 ml of methyl tert-butyl ether by using syringe (12 ml). After stirring for 30 mins, the stir bar was removed. The top clear solution was poured in a Buchner's filter funnel (60 ml), then the polymer suspension placed together in one flask, and stirred for another for 10 mins. The remain suspended solution was filtered out using vacuum filtration. The white-pink product was washed with 100 mL of diethyl ether. The product was placed with the funnel in a vacuum oven at 50° C. overnight. Finally, ˜2.0 g white-pale pink product was obtained.

### Example 27: Preparation of a Calibration Medium Including Two Different Fluorescent Compounds

600 mgs of solvent (20% ethanol and 80% water) were added to 400 mgs of the polymer from Example 26 in a glass vial with an appropriate sized stir bar to agitate at a rate of 500 rpm at 50° C. hot plate overnight. When the resulting formulation was completely dissolved into an uniform solution, free of debris, carbon black was added to create a final concentration of 0.2% carbon black by weight. The mixture was stirred until ready for use.

### Example 28: Preparation of a Calibration Layer Including Two Different Fluorescent Compounds

A Vermes microdispenser (Vermes Microdispensing, Holzkirchen, Germany) was used to dispense 4 spots of approximately 500 nanoliters each into an open channel of each of 5 different microfluidic devices (each without a cover layer). After dispensing, the spots were dried in a drier. Then the cover layer was laminated over each open channel to seal the channel.

The fluorescence intensity of the spots was determined using the configuration shown in FIG. 1 with respect to the optical sensor. The CV of the fluorescence intensities across all 5 strips was 1%. When the identical calibration layers were formed but without using carbon black, the CV of the fluorescence intensities across all 5 strips was 4.7%.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1.-12. (canceled)

13. An optical sensor for detecting a target present in a sample fluid, comprising:
a detection chamber configured to receive the sample fluid;
a polymeric optical sensor medium that (i) is disposed within the detection chamber and has an inner surface configured to contact sample fluid present in the detection chamber, (ii) is at least semi-permeable to the target, (iii) comprises a luminescent reagent that is covalently bound to a polymer of the polymeric optical sensing layer and is configured to generate an optical signal indicative of the presence of the target within the optical sensor medium when the optical sensor medium is irradiated with excitation light from an excitation source, and (iv) comprises an optical reference that is covalently bound to a polymer of the polymeric optical sensing layer and is configured to generate an optical signal generally independent of the presence of the target within the optical sensor medium when the optical sensor medium is irradiated with light from an excitation source, and (v) has a transmittance of about 20% or less at a wavelength of maximum intensity of the excitation light.

14. The optical sensor of claim 13, wherein the optical sensor medium has a thickness in the dry state, of about 75 μm or less along an optical axis of the excitation light from the excitation source.

15. The optical sensor of claim 13, wherein the optical sensor medium has a thickness in the dry state, of at least about 20 μm along an optical axis of the excitation light from the excitation source.

16. The optical sensor of claim 13, wherein the detection chamber comprises a first wall that is transparent at a wavelength of maximum intensity of the excitation light, the optical sensor medium has an outer surface disposed against an inner surface of the first wall of the detection chamber, and an optical axis of the excitation light passes through the first wall into the optical sensor medium.

17. The optical sensor of claim 13, wherein the luminescent reagent comprises a first moiety configured to interact with the target, a second moiety comprising a fluorescent moiety, and a linker by which the luminescent reagent is covalently bound to the polymer and wherein the linker is disposed between the first moiety and the second moiety or is disposed on the first moiety.

18. The optical sensor of claim 17, wherein the first moiety is configured to chelate a target.

19. The optical sensor of claim 13, wherein the optical isolating reagent comprises carbon black, carbon nanostructures, and/or a non-fluorescent dye.

20. (canceled)

21. The optical sensor of claim 13, wherein the sample fluid comprises blood, plasma, or serum.

22. (canceled)

23. The optical sensor of claim 13, wherein the target is selected from the group consisting of Ca++, K+, Na+, or H+, creatinine, lactate, and glucose.

24. A method of detecting a target present in a sample fluid, the method comprising:
contacting a first surface of a polymeric optical sensor medium with the sample fluid, the optical sensor medium being at least semi-permeable to the target and comprising a luminescent reagent covalently bound to a polymer of the polymeric optical sensor medium and configured to generate an optical signal indicative of the presence of the target within the optical sensor medium when the optical sensor medium is irradiated with excitation light from an excitation source and an optical reference covalently bound to a polymer of the polymeric optical sensing layer configured to generate an optical signal generally independent of the presence of the target within the optical sensor when the optical sensor medium is irradiated with excitation light from an excitation source;
irradiating the optical sensor medium with the excitation light along an optical axis that passes into the optical sensor medium through a second surface of the optical sensor medium not in contact with the sample fluid, the optical axis being oriented toward the first surface of the optical sensor medium; and
absorbing at least about 80% of the excitation light within the optical sensor medium prior to the excitation light reaching the first surface of the optical sensor medium.

25.-28. (canceled)

29. The method of claim 24, wherein essentially all of the detected fluorescence arises from the luminescent reagent or the optical reference in the optical sensing layer with the optical isolating reagent.

30. The method of claim 24, wherein the target is selected from the group consisting of Ca++, K+, Na+, or H+, creatinine, lactate, and glucose.

31. The method of claim 24, wherein the optical isolating reagent comprises carbon black, carbon nanostructures, and/or a non-fluorescent dye.

32. The method of claim 24, wherein the optical sensing layer has a thickness, in the dry state, of about 75 μm or less.

33. The method of claim 24, wherein the optical sensing layer has a thickness, in the dry state, of at least about 20 μm.

34. The method of claim 24, wherein the luminescent reagent comprises a first moiety configured to interact with the target, a second moiety comprising a fluorescent moiety, and a linker by which the luminescent reagent is covalently bound to the polymer and wherein the linker is disposed between the first moiety and the second moiety or is disposed on the first moiety.

35. The method of claim 34, wherein the first moiety is configured to chelate a target.
